SEQUENCE LISTING (69 sequences)

SEQ ID NO: 1 - DNA, 789 bp, Euprosthenops australis
ggtccgaattcaggtcaaggaggatatggtggactaggtcaaggagggtatggacaaggtgcaggaagttctgcagccgctgccgccgccgcagcagccgccgcagcaggtggacaaggtggacaaggtcaaggaggatatggacaaggttcaggaggttctgcagccgccgccgccgccgcagcagcagcagcagctgcagcagctggacgaggtcaaggaggatatggccaaggttctggaggtaatgctgctgccgcagccgctgccgccgccgccgccgctgcagcagccggacagggaggtcaaggtggatatggtagacaaagccaaggtgctggttccgctgctgctgctgctgctgctgctgccgctgctgctgctgcaggatctggacaaggtggatacggtggacaaggtcaaggaggttatggtcagagtagtgcttctgcttcagctgctgcgtcagctgctagtactgtagctaattcggtgagtcgcctctcatcgccttccgcagtatctcgagtttcttcagcagtttctagcttggtttcaaatggtcaagtgaatatggcagcgttacctaatatcatttccaacatttcttcttctgtcagtgcatctgctcctggtgcttctggatgtgaggtcatagtgcaagctctactcgaagtcatcactgctcttgttcaaatcgttagttcttctagtgttggatatattaatccatctgctgtgaaccaaattactaatgttgttgctaatgccatggctcaagtaatgggc

SEQ ID NO: 2 - protein, 263 aa, Euprosthenops australis
  DOMAIN (1)..(158): REP fragment
  DOMAIN (159)..(165): Spacer fragment
  DOMAIN (166)..(263): CT fragment
GPNSGQGGYGGLGQGGYGQGAGSSAAAAAAAAAAAAGGQGGQGQGGYGQGSGGSAAAAAAAAAAAAAAAGRGQGGYGQGSGGNAAAAAAAAAAAAAAAGQGGQGGYGRQSQGAGSAAAAAAAAAAAAAAGSGQGGYGGQGQGGYGQSSASASAAASAASTVANSVSRLSSPSAVSRVSSAVSSLVSNGQVNMAALPNIISNISSSVSASAPGASGCEVIVQALLEVITALVQIVSSSSVGYINPSAVNQITNVVANAMAQVMG

SEQ ID NO: 3 - protein, 98 aa, Euprosthenops australis
SRLSSPSAVSRVSSAVSSLVSNGQVNMAALPNIISNISSSVSASAPGASGCEVIVQALLEVITALVQIVSSSSVGYINPSAVNQITNVVANAMAQVMG

SEQ ID NO: 4 - protein, 100 aa, Artificial Sequence
  Consensus sequence derived from known MaSp1 and      MaSp2 proteins
  MISC_FEATURE (1)..(71): Sequence length present in known species      variants
  VARIANT (7)..(7): Glu
SRLSSPQASSRVSSAVSNLVSSGPTNSAALSNTISNVVSQISASNPGLSGCDVLVQALLEVVSALVHILGSSSIGQVNYGSAGQATQIVGQSVAQALGEF

SEQ ID NO: 5 - protein, 1110 aa, Euprosthenops australis
  REPEAT (7)..(19)
  REPEAT (20)..(42)
  REPEAT (43)..(56)
  REPEAT (57)..(70)
  REPEAT (71)..(83)
  REPEAT (84)..(106)
  REPEAT (107)..(120)
  REPEAT (121)..(134)
  REPEAT (135)..(147)
  REPEAT (148)..(170)
  REPEAT (171)..(183)
  REPEAT (184)..(197)
  REPEAT (198)..(211)
  REPEAT (212)..(234)
  REPEAT (235)..(248)
  REPEAT (249)..(265)
  REPEAT (266)..(279)
  REPEAT (280)..(293)
  REPEAT (294)..(306)
  REPEAT (307)..(329)
  REPEAT (330)..(342)
  REPEAT (343)..(356)
  REPEAT (357)..(370)
  REPEAT (371)..(393)
  REPEAT (394)..(406)
  REPEAT (407)..(420)
  REPEAT (421)..(434)
  REPEAT (435)..(457)
  REPEAT (458)..(470)
  REPEAT (471)..(488)
  REPEAT (489)..(502)
  REPEAT (503)..(516)
  REPEAT (517)..(529)
  REPEAT (530)..(552)
  REPEAT (553)..(566)
  REPEAT (567)..(580)
  REPEAT (581)..(594)
  REPEAT (595)..(617)
  REPEAT (618)..(630)
  REPEAT (631)..(647)
  REPEAT (648)..(661)
  REPEAT (662)..(675)
  REPEAT (676)..(688)
  REPEAT (689)..(711)
  REPEAT (712)..(725)
  REPEAT (726)..(739)
  REPEAT (740)..(752)
  REPEAT (753)..(775)
  REPEAT (776)..(789)
  REPEAT (790)..(803)
  REPEAT (804)..(816)
  REPEAT (817)..(839)
  REPEAT (840)..(853)
  REPEAT (854)..(867)
  REPEAT (868)..(880)
  REPEAT (881)..(903)
  REPEAT (904)..(917)
  REPEAT (918)..(931)
  REPEAT (932)..(945)
  REPEAT (946)..(968)
  REPEAT (969)..(981)
  REPEAT (982)..(998)
  REPEAT (999)..(1013)
  REPEAT (1014)..(1027)
  REPEAT (1028)..(1042)
  REPEAT (1043)..(1059)
  REPEAT (1060)..(1073)
  REPEAT (1074)..(1092)
QGAGGNAAAAAAAAAAAAAGQGGQGGYGGLGQGGYGQGAGSSAAAAAAAAAAAAAAGRGQGGYGQGSGGNAAAAAAAAAAAASGQGGQGGQGGQGQGGYGQGAGSSAAAAAAAAAAAAAAGQGQGRYGQGAGGNAAAAAAAAAAAAAGQGGQGGQGGLGQGGYGQGAGSSAAAAAASAAAAAAGRGQGGYGQGAGGNAAAAAAAAAAAAAAGQGGQGGYGGLGQGGYGQGAGSSAAAAAAAAAAAAAGGQGGQGQGRYGQGAGSSAAAAAAAAAAAAAAGQGQGGYGQGAGGNAAAAAAAAAAAAAGQGGQGGQGGLGQGGYGQGAGSSAAAAAAAAAAAAAGRGQGGYGQGAGGNAAAAAAAAAEAAAAGQGGQGGYGGLGQGGYGQGAGSSAAAAAAAAAAAAAGRGQGGYGQGAGGNAAAAAAAAAAAAAAGQGGQGGYGGLGQGGYGQGAGSSAAAAAAAAAAAAAGGQGGQGQGRYGQGAGSSAAAAAAAAAAAAAAGRGQGGYGQGSGGNAAAAAAAAAAAASGQGSQGGQGGQGQGGYGQGAGSSAAAAAAAAAAAAASGRGQGGYGQGAGGNAAAAAAAAAAAAAAGQGGQGGYGGLGQGGYGQGAGSSAAAAAAAAAAAAGGQGGQGQGGYGQGAGSSAAAAAAAAAAAAAAGRGQGGYGQGSGGNAAAAAAAAAAAASGQGGQGGQGGQGQGGYGQGAGSSAAAAAAAAAAAAAAGQGQGGYGQGAGGNAAAAAAAAAAAAAGQGGQGGQGGLGQGGYGQGAGSSAAAAAAAAAAAAAAGRGQGGYGQGVGGNAAAAAAAAAAAAAGQGGQGGQGGLGQGGYGQGAGSSAAAAAAAAAAAAAAGRGQGGYGQGSGGNAAAAAAAAAAAASGQGSQGGQGGQGQGGYGQGAGSSAAAAAAAAAAAAASGRGQGGYGQGAGGNAAAAAAAAAAAAAAGQGGQGGYGGLGQGGYGQGAGSSAAAAAAAAAAAAGGQGGQGQGGYGQGSGGSAAAAAAAAAAAAAAAGRGQGGYGQGSGGNAAAAAAAAAAAAAAAGQGGQGGYGRQSQGAGSAAAAAAAAAAAAAAGSGQGGYGGQGQGGYGQSSASASAAASAASTVANSVS

SEQ ID NO: 6 - protein, 23 aa, Artificial Sequence
  Consensus sequence derived from internal      repeats of Euprosthenops australis MaSp1
  VARIANT (4)..(4): Ser
  VARIANT (8)..(8): Tyr
  VARIANT (11)..(11): Gln
GQGGQGGQGGLGQGGYGQGAGSS

SEQ ID NO: 7 - protein, 17 aa, Artificial Sequence
  Consensus sequence derived from internal      repeats of Euprosthenops australis MaSp1
  VARIANT (9)..(9): Arg
  VARIANT (14)..(14): Ser
  VARIANT (16)..(16): Gly
GQGGQGQGGYGQGAGSS

SEQ ID NO: 8 - protein, 14 aa, Artificial Sequence
  Consensus sequence derived from internal      repeats of Euprosthenops australis MaSp1
  VARIANT (2)..(2): Gln
  VARIANT (6)..(6): Arg
  VARIANT (11)..(11): Ser
  VARIANT (11)..(11): Val
GRGQGGYGQGAGGN

SEQ ID NO: 9 - protein, 10 aa, Artificial Sequence
  cell-binding peptide
  misc_feature: X = any amino acid other than Cys
  DISULFID (1)..(10)
CXXRGDXXXC

SEQ ID NO: 10 - protein, 5 aa, Homo sapiens
IKVAV

SEQ ID NO: 11 - protein, 5 aa, Homo sapiens
YIGSR

SEQ ID NO: 12 - protein, 5 aa, Homo sapiens
EPDIM

SEQ ID NO: 13 - protein, 5 aa, Homo sapiens
NKDIL

SEQ ID NO: 14 - protein, 5 aa, Homo sapiens
GRKRK

SEQ ID NO: 15 - protein, 15 aa, Homo sapiens
KYGAASIKVAVSADR

SEQ ID NO: 16 - protein, 12 aa, Homo sapiens
NGEPRGDTYRAY

SEQ ID NO: 17 - protein, 11 aa, Homo sapiens
PQVTRGDVFTM

SEQ ID NO: 18 - protein, 12 aa, Homo sapiens
AVTGRGDSPASS

SEQ ID NO: 19 - protein, 8 aa, Homo sapiens
TGRGDSPA

SEQ ID NO: 20 - protein, 10 aa, Artificial Sequence
  Modified from Homo sapiens
  DISULFID (1)..(10)
CTGRGDSPAC

SEQ ID NO: 21 - protein, 12 aa, Artificial Sequence
  Synthetic peptide (see Widhe et al., 2013)
GPNSRGDAGAAS

SEQ ID NO: 22 - protein, 10 aa, Homo sapiens
VTGRGDSPAS

SEQ ID NO: 23 - protein, 10 aa, Artificial Sequence
  modified from Homo sapiens
STGRGDSPAS

SEQ ID NO: 24 - DNA, 813 bp, Artificial Sequence
  Fusion protein
ggtccgaattcacgcggcgatgcaggagcggctagcggtcaaggaggatatggtggactaggtcaaggagggtatggacaaggtgcaggaagttctgcagccgctgccgccgccgcagcagccgccgcagcaggtggacaaggtggacaaggtcaaggaggatatggacaaggttcaggaggttctgcagccgccgccgccgccgcagcagcagcagcagctgcagcagctggacgaggtcaaggaggatatggccaaggttctggaggtaatgctgctgccgcagccgctgccgccgccgccgccgctgcagcagccggacagggaggtcaaggtggatatggtagacaaagccaaggtgctggttccgctgctgctgctgctgctgctgctgccgctgctgctgctgcaggatctggacaaggtggatacggtggacaaggtcaaggaggttatggtcagagtagtgcttctgcttcagctgctgcgtcagctgctagtactgtagctaattcggtgagtcgcctctcatcgccttccgcagtatctcgagtttcttcagcagtttctagcttggtttcaaatggtcaagtgaatatggcagcgttacctaatatcatttccaacatttcttcttctgtcagtgcatctgctcctggtgcttctggatgtgaggtcatagtgcaagctctactcgaagtcatcactgctcttgttcaaatcgttagttcttctagtgttggatatattaatccatctgctgtgaaccaaattactaatgttgttgctaatgccatggctcaagtaatgggc

SEQ ID NO: 25 - protein, 271 aa, Artificial Sequence
  Fusion protein
GPNSRGDAGAASGQGGYGGLGQGGYGQGAGSSAAAAAAAAAAAAGGQGGQGQGGYGQGSGGSAAAAAAAAAAAAAAAGRGQGGYGQGSGGNAAAAAAAAAAAAAAAGQGGQGGYGRQSQGAGSAAAAAAAAAAAAAAGSGQGGYGGQGQGGYGQSSASASAAASAASTVANSVSRLSSPSAVSRVSSAVSSLVSNGQVNMAALPNIISNISSSVSASAPGASGCEVIVQALLEVITALVQIVSSSSVGYINPSAVNQITNVVANAMAQVMG

SEQ ID NO: 26 - DNA, 831 bp, Artificial Sequence
  Fusion protein
ggtccgaattcatgcacaggtcgtggtgattctccggcgtgcggatccgctagcggtcaaggaggatatggtggactaggtcaaggagggtatggacaaggtgcaggaagttctgcagccgctgccgccgccgcagcagccgccgcagcaggtggacaaggtggacaaggtcaaggaggatatggacaaggttcaggaggttctgcagccgccgccgccgccgcagcagcagcagcagctgcagcagctggacgaggtcaaggaggatatggccaaggttctggaggtaatgctgctgccgcagccgctgccgccgccgccgccgctgcagcagccggacagggaggtcaaggtggatatggtagacaaagccaaggtgctggttccgctgctgctgctgctgctgctgctgccgctgctgctgctgcaggatctggacaaggtggatacggtggacaaggtcaaggaggttatggtcagagtagtgcttctgcttcagctgctgcgtcagctgctagtactgtagctaattcggtgagtcgcctctcatcgccttccgcagtatctcgagtttcttcagcagtttctagcttggtttcaaatggtcaagtgaatatggcagcgttacctaatatcatttccaacatttcttcttctgtcagtgcatctgctcctggtgcttctggatgtgaggtcatagtgcaagctctactcgaagtcatcactgctcttgttcaaatcgttagttcttctagtgttggatatattaatccatctgctgtgaaccaaattactaatgttgttgctaatgccatggctcaagtaatgggc

SEQ ID NO: 27 - protein, 277 aa, Artificial Sequence
  Fusion protein
  DISULFID (5)..(14)
GPNSCTGRGDSPACGSASGQGGYGGLGQGGYGQGAGSSAAAAAAAAAAAAGGQGGQGQGGYGQGSGGSAAAAAAAAAAAAAAAGRGQGGYGQGSGGNAAAAAAAAAAAAAAAGQGGQGGYGRQSQGAGSAAAAAAAAAAAAAAGSGQGGYGGQGQGGYGQSSASASAAASAASTVANSVSRLSSPSAVSRVSSAVSSLVSNGQVNMAALPNIISNISSSVSASAPGASGCEVIVQALLEVITALVQIVSSSSVGYINPSAVNQITNVVANAMAQVMG

SEQ ID NO: 28 - protein, 267 aa, Artificial Sequence
  Fusion protein
GSGNSGIQGQGGYGGLGQGGYGQGAGSSAAAAAAAAAAAAGGQGGQGQGGYGQGSGGSAAAAAAAAAAAAAAAGRGDGGYGQGSGGNAAAAAAAAAAAAAAAGQGGQGGYGRQSQGAGSAAAAAAAAAAAAAAGSGQGGYGGQGQGGYGQSSASASAAASAASTVANSVSRLSSPSAVSRVSSAVSSLVSNGQVNMAALPNIISNISSSVSASAPGASGCEVIVQALLEVITALVQIVSSSSVGYINPSAVNQITNVVANAMAQVMG

SEQ ID NO: 29 - protein, 267 aa, Artificial Sequence
  Fusion protein
GSGNSGIQGQGGYGGLGQGGYGQGAGSSAAAAAAAAAAAAGGQGGQGQGGYGQGSGGSAAAAAAAAAAAAAAAGRGQGGYGQGSGGNAAAAAAAAAAAAAAAGQGGQGGYGRGDQGAGSAAAAAAAAAAAAAAGSGQGGYGGQGQGGYGQSSASASAAASAASTVANSVSRLSSPSAVSRVSSAVSSLVSNGQVNMAALPNIISNISSSVSASAPGASGCEVIVQALLEVITALVQIVSSSSVGYINPSAVNQITNVVANAMAQVMG

SEQ ID NO: 30 - protein, 272 aa, Artificial Sequence
  Fusion protein
GPNSGRKRKAGAASGQGGYGGLGQGGYGQGAGSSAAAAAAAAAAAAGQGGQGQGGYGQGSGGSAAAAAAAAAAAAAAAGRGQGGYGQGSGGNAAAAAAAAAAAAAAAGQGGQGGYGRQSQGAGSAAAAAAAAAAAAAAGSGQGGYGGQGQGGYGQSSASASAAASAASTVANSVSRLSSPSAVSRVSSAVSSLVSNGQVNMAALPNIISNISSSVSASAPGASGCEVIVQALLEVITALVQIVSSSSVGYINPSAVNQITNVVANAMAQVMG

SEQ ID NO: 31 - protein, 272 aa, Artificial Sequence
  Fusion protein
GPNSIKVAVAGARSGQGGYGGLGQGGYGQGAGSSAAAAAAAAAAAAGQGGQGQGGYGQGSGGSAAAAAAAAAAAAAAAGRGQGGYGQGSGGNAAAAAAAAAAAAAAAGQGGQGGYGRQSQGAGSAAAAAAAAAAAAAAGSGQGGYGGQGQGGYGQSSASASAAASAASTVANSVSRLSSPSAVSRVSSAVSSLVSNGQVNMAALPNIISNISSSVSASAPGASGCEVIVQALLEVITALVQIVSSSSVGYINPSAVNQITNVVANAMAQVMG

SEQ ID NO: 32 - protein, 18 aa, Euprosthenops australis
ASASAAASAASTVANSVS

SEQ ID NO: 33 - protein, 8 aa, Euprosthenops australis
ASAASAAA

SEQ ID NO: 34 - protein, 8 aa, Euprosthenops australis
GSAMGQGS

SEQ ID NO: 35 - protein, 5 aa, Euprosthenops australis
SASAG

SEQ ID NO: 36 - protein, 100 aa, Euprosthenops sp
SRLSSPEASSRVSSAVSNLVSSGPTNSAALSSTISNVVSQIGASNPGLSGCDVLVQALLEVVSALIHILGSSSIGQVNYGSAGQATQLVGQSVYQALGEF

SEQ ID NO: 37 - protein, 98 aa, Euprosthenops australis; identical to SEQ ID NO: 3

SEQ ID NO: 38 - protein, 99 aa, Argiope trifasciata
SRLSSPGAASRVSSAVTSLVSSGGPTNSAALSNTISNVVSQISSSNPGLSGCDVLVQALLEIVSALVHILGSANIGQVNSSGVGRSASIVGQSINQAFS

SEQ ID NO: 39 - protein, 89 aa, Cyrtophora moluccensis
SHLSSPEASSRVSSAVSNLVSSGSTNSAALPNTISNVVSQISSSNPGLSGCDVLVQALLEVVSALIHILGSSSIGQVNYGSAGQATQIV

SEQ ID NO: 40 - protein, 98 aa, Latrodectus geometricus
SALAAPATSARISSHASTLLSNGPTNPASISNVISNAVSQISSSNPGASSCDVLVQALLELVTALLTIIGSSNVGNVNYDSSGQYAQVVSQSVQNAFV

SEQ ID NO: 41 - protein, 98 aa, Latrodectus hesperus
SALSAPATSARISSHASALLSSGPTNPASISNVISNAVSQISSSNPGASACDVLVQALLELVTALLTIIGSSNIGSVNYDSSGQYAQVVTQSVQNVFG

SEQ ID NO: 42 - protein, 93 aa, Macrothele holsti
SHLSSPEASSRVSSAVSNLVSGGSTNSAALPNTISNVVSQISSSNPGLSGCDVLVQALLEVVSALIHILGSSSIGQVDYGSAGQATQIVGQSA

SEQ ID NO: 43 - protein, 98 aa, Nephila clavipes
SRLSSPQASSRVSSAVSNLVASGPTNSAALSSTISNVVSQIGASNPGLSGCDVLIQALLEVVSALIQILGSSSIGQVNYGSAGQATQIVGQSVYQALG

SEQ ID NO: 44 - protein, 89 aa, Nephila pilipes
SRLSSPEASSRVSSAVSNLVSSGPTNSAALSNTISNVVSQISSSNPGLSGCDVLVQALLEVVSALIHILGSSSIGQVNYGSAGQATQIV

SEQ ID NO: 45 - protein, 87 aa, Nephila madagascariensis
SRLSSPQASSRVSSAVSNLVASGPTNSAALSSTISNAVSQIGASNPGLSGCDVLIQALLEVVSALIHILGSSSIGQVNYGSAGQATQ

SEQ ID NO: 46 - protein, 87 aa, Nephila senegalensis
SRLSSPEASSRVSSAVSNLVSSGPTNSAALSSTISNVVSQIGASNPGLSGCDVLIQALLEVVSALVHILGSSSIGQVNYGSAGQATQ

SEQ ID NO: 47 - protein, 89 aa, Octonoba varians
SRLSSPEASSRVSSAVSNLVSSGPTNSAALSNTISNVVSQISSSNPGLSGCDVLVQALLEVVSAPIHILGSSSIGQVNYGSAGQATQIV

SEQ ID NO: 48 - protein, 89 aa, Psechrus sinensis
SRLSSPEASSRVSSAVSNLVSSGPTNSAALPNTISNVVSQISSSNPGLSGCDVLVQALLEVVSALIHILGSSSIGQVNYGSAGQATQIV

SEQ ID NO: 49 - protein, 88 aa, Tetragnatha kauaiensis
SLLSSPASNARISSAVSALASGAASGPGYLSSVISNVVSQVSSNSGGLVGCDTLVQALLEAAAALVHVLASSSGGQVNLNTAGYTSQL

SEQ ID NO: 50 - protein, 88 aa, Tetragnatha versicolor
SRLSSPASNARISSAVSALASGGASSPGYLSSIISNVVSQVSSNNDGLSGCDTVVQALLEVAAALVHVLASSNIGQVNLNTAGYTSQL

SEQ ID NO: 51 - protein, 89 aa, Araneus bicentenarius
SRLSSSAASSRVSSAVSSLVSSGPTTPAALSNTISSAVSQISASNPGLSGCDVLVQALLEVVSALVHILGSSSVGQINYGASAQYAQMV

SEQ ID NO: 52 - protein, 97 aa, Argiope amoena
RLSSPQASSRVSSAVSTLVSSGPTNPASLSNAIGSVVSQVSASNPGLPSCDVLVQALLEIVSALVHILGSSSIGQINYSASSQYARLVGQSIAQALG

SEQ ID NO: 53 - protein, 82 aa, Argiope aurantia
SRLSSPQASSRVSSAVSTLVSSGPTNPAALSNAISSVVSQVSASNPGLSGCDVLVQALLELVSALVHILGSSSIGQINYAAS

SEQ ID NO: 54 - protein, 98 aa, Argiope trifasciata
SRLSSPQASSRVSSAVSTLVSSGPTNPASLSNAISSVVSQVSSSNPGLSGCDVLVQALLEIVSALVHILGSSSIGQINYAASSQYAQLVGQSLTQALG

SEQ ID NO: 55 - protein, 89 aa, Gasteracantha mammosa
SRLSSPQAGARVSSAVSALVASGPTSPAAVSSAISNVASQISASNPGLSGCDVLVQALLEIVSALVSILSSASIGQINYGASGQYAAMI

SEQ ID NO: 56 - protein, 90 aa, Latrodectus geometricus
SALSSPTTHARISSHASTLLSSGPTNSAAISNVISNAVSQVSASNPGSSSCDVLVQALLELITALISIVDSSNIGQVNYGSSGQYAQMVG

SEQ ID NO: 57 - protein, 98 aa, Latrodectus hesperus
SALSSPTTHARISSHASTLLSSGPTNAAALSNVISNAVSQVSASNPGSSSCDVLVQALLEIITALISILDSSSVGQVNYGSSGQYAQIVGQSMQQAMG

SEQ ID NO: 58 - protein, 97 aa, Nephila clavipes
SRLASPDSGARVASAVSNLVSSGPTSSAALSSVISNAVSQIGASNPGLSGCDVLIQALLEIVSACVTILSSSSIGQVNYGAASQFAQVVGQSVLSAF

SEQ ID NO: 59 - protein, 82 aa, Nephila madagascariensis
SRLASPDSGARVASAVSNLVSSGPTSSAALSSVISNAVSQIGASNPGLSGCDVLIQALLEIVSACVTILSSSSIGQVNYGAA

SEQ ID NO: 60 - protein, 82 aa, Nephila senegalensis
  misc_feature (35)..(35): Xaa can be any naturally occurring amino acid
  misc_feature (56)..(56): Xaa can be any naturally occurring amino acid
SRLASPDSGARVASAVSNLVSSGPTSSAALSSVIXNAVSQIGASNPGLSGCDVLIXALLEIVSACVTILSSSSIGQVNYGAA

SEQ ID NO: 61 - protein, 71 aa, Dolomedes tenebrosus
SRLSSPEAASRVSSAVSSLVSNGQVNVDALPSIISNLSSSISASATTASDCEVLVQVLLEVVSALVQIVCS

SEQ ID NO: 62 - protein, 97 aa, Dolomedes tenebrosus
SRLSSPQAASRVSSAVSSLVSNGQVNVAALPSIISSLSSSISASSTAASDCEVLVQVLLEIVSALVQIVSSANVGYINPEASGSLNAVGSALAAAMG

SEQ ID NO: 63 - protein, 93 aa, Araneus diadematus
NRLSSAGAASRVSSNVAAIASAGAAALPNVISNIYSGVLSSGVSSSEALIQALLEVISALIHVLGSASIGNVSSVGVNSALNAVQNAVGAYAG

SEQ ID NO: 64 - protein, 98 aa, Araneus diadematus
SRLSSPSAAARVSSAVSLVSNGGPTSPAALSSSISNVVSQISASNPGLSGCDILVQALLEIISALVHILGSANIGPVNSSSAGQSASIVGQSVYRALS

SEQ ID NO: 65 - protein, 98 aa, Araneus diadematus
SRLSSPAASSRVSSAVSSLVSSGPTKHAALSNTISSVVSQVSASNPGLSGCDVLVQALLEVVSALVSILGSSSIGQINYGASAQYTQMVGQSVAQALA

SEQ ID NO: 66 - protein, 94 aa, Araneus diadematus
SVYLRLQPRLEVSSAVSSLVSSGPTNGAAVSGALNSLVSQISASNPGLSGCDALVQALLELVSALVAILSSASIGQVNVSSVSQSTQMISQALS

SEQ ID NO: 67 - protein, 10 aa, Homo sapiens
STGRGDSPAV

SEQ ID NO: 68 - protein, 93 aa, Araneus ventricosus
NRLSSAEAASRVSSNIAAIASGGASALPSVISNIYSGVVASGVSSNEALIQALLELLSALVHVLSSASIGNVSSVGVDSTLNVVQDSVGQYVG

SEQ ID NO: 69 - protein, 272 aa, Artificial Sequence
  Fusion protein
GPNSCTGRGDSPACGSASGQGGYGGLGQGGYGQGAGSSAAAAAAAAAAAAGGQGGQGQGGYGQGSGGSAAAAAAAAAAAAAAAGRGQGGYGQGSGGNAAAAAAAAAAAAAAAGQGGQGGYGRQSQGAGSAAAAAAAAAAAAAAGSGQGGYGGQGQGGYGQSSASASAAASAAGSYAGAVNRLSSAEAASRVSSNIAAIASGGASALPSVISNIYSGVVASGVSSNEALIQALLELLSALVHVLSSASIGNVSSVGVDSTLNVVQDSVGQYVG

(19) United States
(12) Patent Application Publication
(10) Pub. No.: US 2019/0144819 A1
(43) Pub. Date: May 16, 2019

(54) INTEGRATED CELLS

(71) Applicant: SPIBER TECHNOLOGIES AB, Stockholm (SE)

(72) Inventors: My HEDHAMMAR, Stockholm (SE); Mona WIDHE, Uppsala (SE); Ulrika JOHANSSON, Uppsala (SE)

(73) Assignee: SPIBER TECHNOLOGIES AB, Stockholm (SE)

(21) Appl. No.: 16/077,179

(22) PCT Filed: Feb. 10, 2017
(86) PCT No.: PCT/EP2017/053084

§ 371 Date: Aug. 10, 2018

(30) Foreign Application Priority Data
Feb. 12, 2016 (EP) 16155494.4
Oct. 18, 2016 (EP) 16194431.9

(51) Int. Cl. C12N 5/00; C07K 14/435

(52) U.S. Cl. CPC C12N 5/0068; C12N 2533/90; C12N 2533/50; C07K 14/43586

(57) ABSTRACT

A cell scaffold material is manufactured by providing an aqueous solution of a silk protein capable of assembling into a water-insoluble macrostructure. The silk protein is mixed with eukaryotic cells, and the silk protein is assembled into a water-insoluble macrostructure in the presence of the cells, thereby forming a scaffold material for cultivating the cells. The cells can be grown integrated with the scaffold material under conditions suitable for cell culture.

### TECHNICAL FIELD OF THE INVENTION

The present invention relates to the fields of eukaryotic cell culture and tissue engineering, and provides methods and a cell scaffold material for culture of eukaryotic cells, wherein a polymer of a silk protein, such as a fibroin or a spider silk protein, is used as a cell scaffold material.

### BACKGROUND TO THE INVENTION

The fundamental concept of tissue engineering is to combine different components, such as living cells, biomaterial and bioactive factors, to form engineered tissue constructs. Traditional tissue engineering strategies typically employ a “top-down” approach, in which cells are seeded on a polymeric scaffold. The material must then contain large pores with high interconnectivity to allow subsequent cell infiltration. In order to allow a high porosity without collapse, the material has to have thick and/or stiff walls, which leads to poor cell compatibility and low flexibility when the cells are about to expand.

As alternative, the “bottom-up” tissue engineering approach has been initiated lately. A bottom-up approach relies on the assembly of a matrix from smaller components or modules together with the cells. For example, this can be achieved by 3D printing of hydrogels containing cells. However, one major drawback of hydrogels is the lack of mechanical strength, which restricts their use to soft tissue engineering. The processes used for formulation of stronger synthetic matrices are typically dependent on harsh conditions such as melting or organic solvents, and hence not compatible with cell viability. Moreover, synthetic material typically gets much stiffer than what is suitable to match mammalian tissue. The natural extracellular matrix (ECM) that surrounds mammalian cells in tissue consists of fibers (e.g. collagen and elastin) composed of modified proteins that are demanding to produce synthetically, and in vitro mimicry of their mechanical properties has so far not been accomplished. Also other organisms use protein fibers as support; the strongest being silk threads spun by spiders. Apart from outstanding strength, spider silk has very attractive properties such as elasticity and biocompatibility.

Spiders have up to seven different glands which produce a variety of silk types with different mechanical properties and functions. Dragline silk, produced by the major ampullate gland, is the toughest fiber, and on a weight basis it outperforms man-made materials, such as tensile steel. The properties of dragline silk are attractive in development of new materials for medical or technical purposes, e.g. as scaffolds for cell culture.

Dragline silk consists of two main polypeptides, mostly referred to as major ampullate spidroin (MaSp) 1 and 2, but e.g. as ADF-3 and ADF-4 in Araneus diadematus. These proteins have molecular masses in the range of 200-720 kDa. The genes coding for dragline proteins of Latrodectus hesperus are the only ones that have been completely characterized, and the MaSp1 and MaSp2 genes encode 3129 and 3779 amino acids, respectively (Ayoub NA et al. PLoS ONE 2(6): e514, 2007). The properties of dragline silk polypeptides are discussed in Huemmerich, D. et al. Curr. Biol. 14, 2070-2074 (2004).

Spider dragline silk proteins, or MaSps, have a tripartite composition; a non-repetitive N-terminal domain, a central repetitive region comprised of many iterated poly-Ala/Gly segments, and a non-repetitive C-terminal domain. It is generally believed that the repetitive region forms intermolecular contacts in the silk fibers, while the precise functions of the terminal domains are less clear. It is also believed that in association with fiber formation, the repetitive region undergoes a structural conversion from random coil and α-helical conformation to β-sheet structure. The C-terminal region of spidroins is generally conserved between spider species and silk types. The N-terminal domain of spider silks is the most conserved region (Rising, A. et al. Biomacromolecules 7, 3120-3124 (2006)).

WO 07/078239 and Stark, M. et al., Biomacromolecules 8, 1695-1701, (2007) disclose a miniature spider silk protein consisting of a repetitive fragment with a high content of Ala and Gly and a C-terminal fragment of a protein, as well as soluble fusion proteins comprising the spider silk protein. The spider silk protein is spontaneously transformed into a coherent and water insoluble macrostructure, e.g. an ordered polymer such as a fiber, upon subjection to an interface such as air:water. The miniature spider silk protein unit is sufficient and necessary for the fiber formation. Cells from an immortalized cell line is added onto the pre-formed, macroscopic spider silk fiber and allowed to grow.

Hedhammar, M. et al., Biochemistry 47, 3407-3417, (2008) study the thermal, pH and salt effects on the structure and aggregation and/or polymerisation of recombinant N- and C-terminal spidroin domains and a repetitive spidroin domain containing four poly-Ala and -Gly rich co-blocks.

WO 2011/129756 discloses methods and a cell scaffold material based on a miniature spider silk protein for eukaryotic cell culture. The protein may contain various short (3-5 amino acid residues) cell-binding peptides. Various cell types are added onto the pre-formed cell scaffold material.

WO 2012/055854 discloses manufacture of a cell scaffold material comprising a recombinant protein which is a fusion protein between a spider silk proteins and a longer (>30 amino acid residues), non-spidroin polypeptide or protein with desirable binding properties. Cells are added onto the pre-formed cell scaffold material and cultivated.

WO 2015/036619 and Widhe, M. et al., Biomaterials 74:256-266 (2016) disclose further miniature spider silk proteins with useful cell-binding peptides. Again, various cell types are added onto the pre-formed cell scaffold material.

Johansson et al., PLOS ONE 10(6): e0130169 (2015) discloses formulation of a spider silk protein into various physical formats. Subsequently, pancreatic mouse islets were placed on top of the spider silk matrices and allowed to adhere.

Despite these advances in the field, there is still a need for new cell scaffolds in the field. In particular, there is a need in the field for a mechanically robust, three-dimensional scaffold for cultivation of integrated eukaryotic cells and use in tissue engineering.

### SUMMARY OF THE INVENTION

It is an object of the present invention to provide a cell scaffold with improved cell compatibility and flexibility when the cells are about to expand.

It is also an object of the present invention to provide a cell scaffold which achieves a more tissue-like spreading of cultivated cells.

It is an object of the present invention to provide a cell scaffold with high seeding efficiency, yielding quickly and viably adhered cells.

It is a further object of the present invention to provide a cell scaffold with sufficient mechanical strength and suitable stiffness for mammalian tissue engineering.

It is also an object of the present invention to provide a process for providing a cell scaffold under conditions which are compatible with cell viability.

It is yet another object of the present invention to provide a cell scaffold wherein cells are integrated throughout the cell scaffold material.

It is also an object of the present invention to provide a method which allows for co-cultures of several cell types within the cell scaffolds.

For these and other objects that will be evident from the following disclosure, the present invention provides according to a first aspect a method for the cultivation of eukaryotic cells, comprising the steps:
(a) providing an aqueous solution of a silk protein capable of assembling into a water-insoluble macrostructure, wherein the silk protein optionally contains a cell-binding motif;
(b) preparing an aqueous mixture of a sample of the eukaryotic cells with the silk protein, wherein the silk protein remains dissolved in the aqueous mixture;
(c) allowing the silk protein to assemble into a water-insoluble macrostructure in the presence of the eukaryotic cells, thereby forming a scaffold material for cultivating the eukaryotic cells; and
(d) maintaining the eukaryotic cells within the scaffold material under conditions suitable for cell culture.

In a preferred variant of the method for the cultivation of eukaryotic cells, the silk protein is a spider silk protein.

The invention is based on the inventive insight that dispersed eukaryotic cells can be added to the silk protein solution before assembly of the silk proteins into a water-insoluble macrostructure, and thereby be integrated throughout the silk-like material during the mild self-assembly process. This is in contrast to the prior art cell cultivation methods, where cells have been added onto pre-formed silk macrostructures.

Advantageously, formulation of macrostructures with integrated cells provides a high seeding efficiency, yielding quickly and viably adhered cells.

Compared to cultivation in hydrogels, cells attain a more tissue-like spreading when integrated into silk scaffolds employing the methods according to the invention.

As demonstrated herein, it is not critical which specific spider silk protein is utilized in the present invention. The silk protein is preferably a fibroin, such as a silkworm fibroin, or a spider silk protein.

The present invention provides according to a second aspect a process for manufacturing a cell culture product comprising (i) a scaffold material for cultivating eukaryotic cells; and (ii) eukaryotic cells, which are growing integrated with the scaffold material, comprising the steps:
(a) providing an aqueous solution of a silk protein capable of assembling into a water-insoluble macrostructure, wherein the silk protein optionally contains a cell-binding motif;
(b) preparing an aqueous mixture of a sample of the eukaryotic cells with the silk protein, wherein the silk protein remains dissolved in the aqueous mixture; and
(c) allowing the silk protein to assemble into a water-insoluble macrostructure in the presence of the eukaryotic cells, thereby forming the scaffold material for cultivating the eukaryotic cells.

In a preferred variant of the process for manufacturing a cell culture product, the silk protein is a spider silk protein.

According to a third aspect, the present invention provides a cell culture product comprising (i) a scaffold material for cultivating eukaryotic cells, which is a water-insoluble macrostructure of a silk protein capable of assembling into a water-insoluble macrostructure, wherein the silk protein optionally contains a cell-binding motif; and (ii) eukaryotic cells, which are growing integrated with the scaffold material.

In a preferred variant of the cell culture product, the silk protein is a spider silk protein.

In preferred embodiments, the cell culture product is obtainable or obtained by the manufacturing process according to the invention.

The present invention provides according to a fourth aspect a novel use of a silk protein capable of assembling into a water-insoluble macrostructure in the formation of a scaffold material for cultivating eukaryotic cells in the presence of said cells; wherein the scaffold material is a water-insoluble macrostructure of the silk protein; and wherein the silk protein optionally contains a cell-binding motif.

In a preferred variant of the use, the silk protein is a spider silk protein.

In some preferred embodiments of these and other aspects of the invention, the macrostructure is brought into a shape selected from fiber, foam, film, fiber mesh, capsules and nets, preferably fiber or foam.

In certain preferred embodiments of these and other aspects of the invention, the eukaryotic cells are selected from mammalian cells, preferably selected from primary cells and cell lines, such as endothelical cells, fibroblasts, keratinocytes, skeletal muscle satellite cells, skeletal muscle myoblasts, smooth muscle cells, umbilical vein endothelial cells, Schwann cells, pancreatic β-cells, pancreatic islet cells, hepatocytes and glioma-forming cells; and stem cells, such as mesenchymal stem cells; or a combination of at least two different mammalian cell types.

In certain preferred embodiments of the present invention, the silk protein is a fibroin, such as a silkworm fibroin.

In some preferred embodiments of the present invention, the silk protein is a spider silk protein. In some preferred embodiments of these and other aspects of the invention, the spider silk protein is comprising, or consisting of, the protein moieties REP and CT, wherein REP is a repetitive fragment of from 70 to 300 amino acid residues, selected from the group consisting of L(AG)ₙL, L(AG)ₙAL, L(GA)ₙL, and L(GA)ₙGL, wherein n is an integer from 2 to 10; each individual A segment is an amino acid sequence of from 8 to 18 amino acid residues, wherein from 0 to 3 of the amino acid residues are not Ala, and the remaining amino acid residues are Ala; each individual G segment is an amino acid sequence of from 12 to 30 amino acid residues, wherein at least 40% of the amino acid residues are Gly; and each individual L segment is a linker amino acid sequence of from 0 to 30 amino acid residues; and CT is a fragment of from 70 to 120 amino acid residues, having at least 70% identity to SEQ ID NO: 3 or SEQ ID NO: 68; and wherein the optional cell-binding motif is arranged either terminally in the spider silk protein, or between the moieties, or within any of the moieties, preferably terminally in the spider silk protein.

In certain preferred embodiments of these and other aspects of the invention, the silk protein contains a cell-binding motif, such as a cell-binding motif selected from RGD, IKVAV (SEQ ID NO: 10), YIGSR (SEQ ID NO: 11), EPDIM (SEQ ID NO: 12), NKDIL (SEQ ID NO: 13), GRKRK (SEQ ID NO: 14), KYGAASIKVAVSADR (SEQ ID NO: 15), NGEPRGDTYRAY (SEQ ID NO: 16), PQVTRGDVFTM (SEQ ID NO: 17), AVTGRGDSPASS (SEQ ID NO: 18), TGRGDSPA (SEQ ID NO: 19), CTGRGDSPAC (SEQ ID NO: 20) and FN_{cc} (SEQ ID NO: 9); and preferably from FN_{cc}, GRKRK, IKVAV, RGD and CTGRGDSPAC, more preferably FN_{cc} and CTGRGDSPAC; wherein FN_{cc} is C¹X¹X²RGDX³X⁴X⁵C²; wherein each of X¹, X², X³, X⁴ and X⁵ are independently selected from natural amino acid residues other than cysteine; and C¹ and C² are connected via a disulphide bond.

### BRIEF DESCRIPTION OF THE DRAWINGS

FIG. 1 shows a sequence alignment of spidroin C-terminal domains.

FIG. 2 shows spider silk constructs with cell-binding motifs derived from fibronectin.

FIG. 3 shows formulation of silk scaffolds with integrated cells.

FIG. 4 shows metabolic activity of cells within silk scaffolds.

FIG. 5 shows viability of cells within silk scaffolds.

FIG. 6 shows spreading of cells within silk scaffolds.

FIG. 7 shows distribution of cells within silk scaffolds.

FIG. 8 shows mechanical properties of silk fibers with cells.

FIG. 9 shows immunofluorescence staining of collagen type I in fibroblasts grown on silk scaffolds.

FIG. 10 shows immunofluorescence staining of myotube formation in Hsk cells grown on silk fibers.

FIG. 11 shows presence of several cell types co-cultured within silk scaffolds.

FIG. 12 shows that islet-like clusters are functional within silk scaffolds.

FIG. 13 shows in vivo imaging of silk scaffolds with cells.

FIG. 14 shows cell distribution within silk fibers.

FIG. 15 shows cell distribution within silk foam.

FIG. 16 shows growth curves of proliferating cells within silk foams.

FIG. 17 shows staining of live cells integrated within silk foams.

FIG. 18 shows growth curves of proliferating cells within silk fibers.

FIG. 19 shows staining of live cells integrated within silk fibers.

FIG. 20 shows growth curves of proliferating cells within silk films.

FIG. 21 shows images of live cells integrated within silk films and foams.

FIG. 22 shows micrographs of cells integrated within silk films and their crystal violet absorption.

FIG. 23 shows stem cells differentiated into the adipogenic and osteogenic linages, respectively.

FIG. 24 shows relative gene expression of neuronal progenitor markers in differentiated stem cells.

### LIST OF APPENDED SEQUENCES

SEQ ID NO:
1 RepCT (4RepCT, WT) (DNA)
2 RepCT (4RepCT, WT)
3 CT
4 consensus CT sequence
5 repetitive sequence from Euprosthenops australis MaSp1
6 consensus G segment sequence 1
7 consensus G segment sequence 2
8 consensus G segment sequence 3
9 FN_{cc}
10 IKVAV
11 YIGSR
12 EPDIM
13 NKDIL
14 GRKRK
15 KYGAASIKVAVSADR
16 NGEPRGDTYRAY
17 PQVTRGDVFTM
18 AVTGRGDSPASS
19 TGRGDSPA
20 CTGRGDSPAC
21 GPNSRGDAGAAS
22 VTGRGDSPAS
23 STGRGDSPAS
24 RGD-4RepCT, Widhe et al. (2013) (DNA)*
25 RGD-4RepCT, Widhe et al. (2013)*
26 FN_{cc}-4RepCT (DNA)
27 FN_{cc}-4RepCT
28 2RepRGD2RepCT (2R)
29 3RepRGD1RepCT (3R)
30 GRKRK-4RepCT
31 IKVAV-4RepCT
32 Linker peptide 1
33 Linker peptide 2
34 Linker peptide 3
35 Linker peptide 4

SEQ ID NO:
36 CT Euprosthenops sp MaSp1
37 CT Euprosthenops australis MaSp1
38 CT Argiope trifasciata MaSp1
39 CT Cyrtophora moluccensis Sp1
40 CT Latrodectus geometricus MaSp1
41 CT Latrodectus hesperus MaSp1
42 CT Macrothele holsti Sp1
43 CT Nephila clavipes MaSp1
44 CT Nephila pilipes MaSp1
45 CT Nephila madagascariensis MaSp1
46 CT Nephila senegalensis MaSp1
47 CT Octonoba varians Sp1
48 CT Psechrus sinensis Sp1
49 CT Tetragnatha kauaiensis MaSp1
50 CT Tetragnatha versicolor MaSp1
51 CT Araneus bicentenarius Sp2
52 CT Argiope amoena MaSp2
53 CT Argiope aurantia MaSp2
54 CT Argiope trifasciata MaSp2
55 CT Gasteracantha mammosa MaSp2
56 CT Latrodectus geometricus MaSp2
57 CT Latrodectus hesperus MaSp2
58 CT Nephila clavipes MaSp2
59 CT Nephila madagascariensis MaSp2
60 CT Nephila senegalensis MaSp2
61 CT Dolomedes tenebrosus Fb1
62 CT Dolomedes tenebrosus Fb2
63 CT Araneus diadematus ADF-1
64 CT Araneus diadematus ADF-2
65 CT Araneus diadematus ADF-3
66 CT Araneus diadematus ADF-4
67 STGRGDSPAV (FN1011)
68 CT Aranaeus ventricosus MiSp
69 FN_{cc}-RepCT_{MiSp}

* Widhe M et al., Biomaterials 34(33): 8223-8234 (2013)

### DETAILED DESCRIPTION OF THE INVENTION

Tissues are built up of cells integrated in a composite material, called the extracellular matrix (ECM). The ECM provides physical 3D support and also specific sites for cell anchorage. We have developed a recombinant silk protein functionalized with a motif from the ECM protein fibronectin (FN), which enhance the cell supportive capacity of FN-silk formed thereof. A mild self-assembly process can be used to accomplish various formats of spider silk scaffolds, including foam, fiber and film. The mild self-assembly process is surprisingly also useful to accomplish various formats of fibroin silk, including foam, fiber and film.

Acute injuries and trauma where tissue loss and failure are large causes repair process problems due to loss of guiding extracellular matrix. The healing process is not sufficient and can be life-threatening in case of life support organs such as the liver. A liver has a unique ability to self-renewal and if the liver has the chance and time it can regenerate. The recombinant spider silk could give the support to liver failures by providing a supporting scaffold for the patients' own liver cells that have survived. This could give the liver cells a chance to regenerate and repair and become a personalized liver transplant.

The co-formulation of silk combined with cells from a specific tissue (normal or cancer) could also develop a 3D in vitro platform for disease modeling, drug discovery and toxicology. Cancer treatment is aiming for personal medicine due to the complexity of the cancer disease. A biomimetic 3D culture of co-formulated cancer and recombinant spider silk is one example where it could be possible to screen the cancer progress and develop cancer specific treatment—a personalized method to target and demolish cancer.

The present invention is based on the insight that dispersed mammalian cells can be added to a silk protein solution before assembly thereof into water-insoluble ordered polymers or macrostructures, and thereby be integrated throughout the silk-like material. A collection of various mammalian cell types (from mouse and human) have been successfully been integrated into various silk formats, including fiber, foam and film. The silk protein is a fibroin or a spider silk protein. The proliferative capacity of the cells was maintained through more than two weeks within the spider silk scaffolds, with some variability of when confluence was reached depending on the cell type. The viability was high (>80%) for all cell types investigated, with confirmed viability in the innermost part of the materials. The observed cell infiltration is highly advantageous for the formation of engineered tissue constructs.

It is demonstrated herein that formulation of macrostructures, preferably films and foams, with integrated cells provides a high seeding efficiency, yielding quickly and viably adhered cells. Elongated cells with filamentous actin and defined focal adhesion points confirm proper cell attachment within the scaffolds. Cryosectioning was used to further confirm presence of cells within the deepest parts of the materials. Tensile testing of cell-containing spider silk fibers was performed under physiological-like conditions, to investigate the mechanical properties. In vivo imaging of cell-containing spider silk scaffolds transplanted into the anterior eye chamber confirms maintenance of cells for 4 weeks in vivo.

Compared to cultivation in hydrogels, cells attain a more tissue-like spreading when integrated into silk scaffolds employing the methods according to the invention.

Most native tissue types consist of several cell types organized together in a complex three-dimensional arrangement with extracellular matrix surrounding the cells and keeping them together. In order to replicate this in engineered tissue constructs it is therefore of importance to achieve co-cultures within the scaffolds. With the herein described method for formulation of cell containing silk scaffolds it is practically very easy to combine several cell types.

According to a first aspect, there is provided a method for the cultivation of eukaryotic cells. The method is preferably carried out in vitro.

The method is comprising the steps:

(a) providing an aqueous solution of a silk protein capable of assembling into a water-insoluble macrostructure, wherein the silk protein optionally contains a cell-binding motif;

(b) preparing an aqueous mixture of a sample of the eukaryotic cells with the silk protein, wherein the silk protein remains dissolved in the aqueous mixture;

(c) allowing the silk protein to assemble into a water-insoluble macrostructure in the presence of the eukaryotic cells, thereby forming a scaffold material for cultivating the eukaryotic cells; and

(d) maintaining the eukaryotic cells within the scaffold material under conditions suitable for cell culture.

It is preferred that the eukaryotic cells are mammalian cells, and preferably human cells, including primary cells, cell lines and stem cells. Useful examples of primary cells and cell lines include endothelical cells, fibroblasts, keratinocytes, skeletal muscle satellite cells, skeletal muscle myoblasts, smooth muscle cells, umbilical vein endothelial cells, Schwann cells, pancreatic β-cells, pancreatic islet cells, hepatocytes and glioma-forming cells. The stem cells are preferably human pluripotent stem cells (hPSCs), such as embryonic stem cells (ESC) and induced pluripotent cells (iPS). Useful examples of stem cells include mesenchymal stem cells. The cells may also preferably be a combination of at least two different mammalian cell types, such as those set out above.

In the first step, an aqueous solution of a silk protein capable of assembling into a water-insoluble macrostructure is provided. The composition of the aqueous solution is not critical, but it is generally preferred to use a mild aqueous buffer, e.g. a phosphate buffer with a low or intermediate ion strength and a pH in the range of 6-8. The aqueous solution preferably contains no organic solvents, such as hexafluoroisopropanol, DMSO, and the like.

In certain preferred embodiments of the present invention, the silk protein is a fibroin. Fibroin is present in silk created by spiders, moths, such as silkworms, and other insects. Preferred fibroins are derived from the genus Bombyx, and preferably from the silkworm of Bombyx mori.

In certain preferred embodiments of the present invention, the silk protein is a spider silk protein. The terms “spidroins” and “spider silk proteins” are used interchangeably throughout the description and encompass all known spider silk proteins, including major ampullate spider silk proteins which typically are abbreviated “MaSp”, or “ADF” in the case of Araneus diadematus. These major ampullate spider silk proteins are generally of two types, 1 and 2. These terms furthermore include non-natural proteins with a high degree of identity and/or similarity to the known spider silk proteins.

The silk protein optionally contains a cell-binding motif (CBM). The optional cell-binding motif is arranged either terminally in the silk protein or within the silk protein, preferably N-terminally or C-terminally in the silk protein.

Upon assembly into a macrostructure, the silk protein provides an internal solid support activity for the cells. For avoidance of doubt, the term “macrostructure” refers to a coherent form of the silk protein, typically an ordered polymer, such as a fiber, foam or film, and not to unordered aggregates or precipitates of the same protein. When the silk protein further contains a cell-binding motif, the resulting macrostructure harbors both a desired selective cell-binding activity in the cell-binding motif and an internal solid support activity in the silk protein fragment. The binding activity of the silk protein is maintained when it is structurally rearranged to form polymeric, solid structures. These macrostructures also provide a high and predictable density of the cell-binding motif. The way biomaterials functionalized with e.g. RGD stimulate different cell responses is not only affected by the type of RGD motif used, but also the resulting surface concentrations of ligands. Since the rather small silk proteins used in the present study self-assemble into multilayers where each molecule carries an RGD motif, a dense surface presentation is expected. However, if a sparser surface concentration is desired, any possible surface density can be achieved simply by mixing silk proteins with and without the cyclic RGD cell-binding motif disclosed herein at different ratios, thereby directing the cellular response of interest.

The cell-binding motif may for example comprise an amino acid sequence selected from the group consisting of RGD, IKVAV (SEQ ID NO: 10), YIGSR (SEQ ID NO: 11), EPDIM (SEQ ID NO: 12) and NKDIL (SEQ ID NO: 13). RGD, IKVAV and YIGSR are general cell-binding motifs, whereas EPDIM and NKDIL are known as keratinocyte-specific motifs that may be particularly useful in the context of cultivation of keratinocytes. Other useful cell-binding motifs include GRKRK from tropoelastin (SEQ ID NO: 14), KYGAASIKVAVSADR (laminin derived, SEQ ID NO: 15), NGEPRGDTYRAY (from bone sialoprotein, SEQ ID NO: 16), PQVTRGDVFTM (from vitronectin, SEQ ID NO: 17), AVTGRGDSPASS (from fibronectin, SEQ ID NO: 18), TGRGDSPA (SEQ ID NO: 19) and FN_{cc}, such as CTGRGDSPAC (SEQ ID NO: 20).

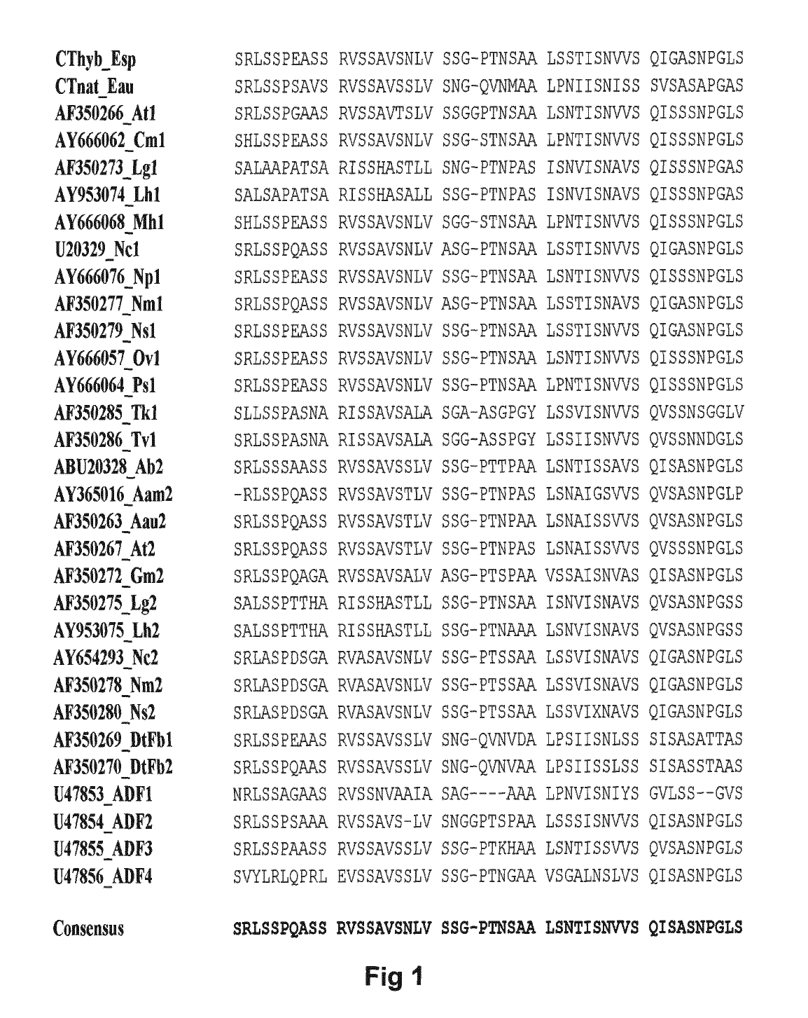

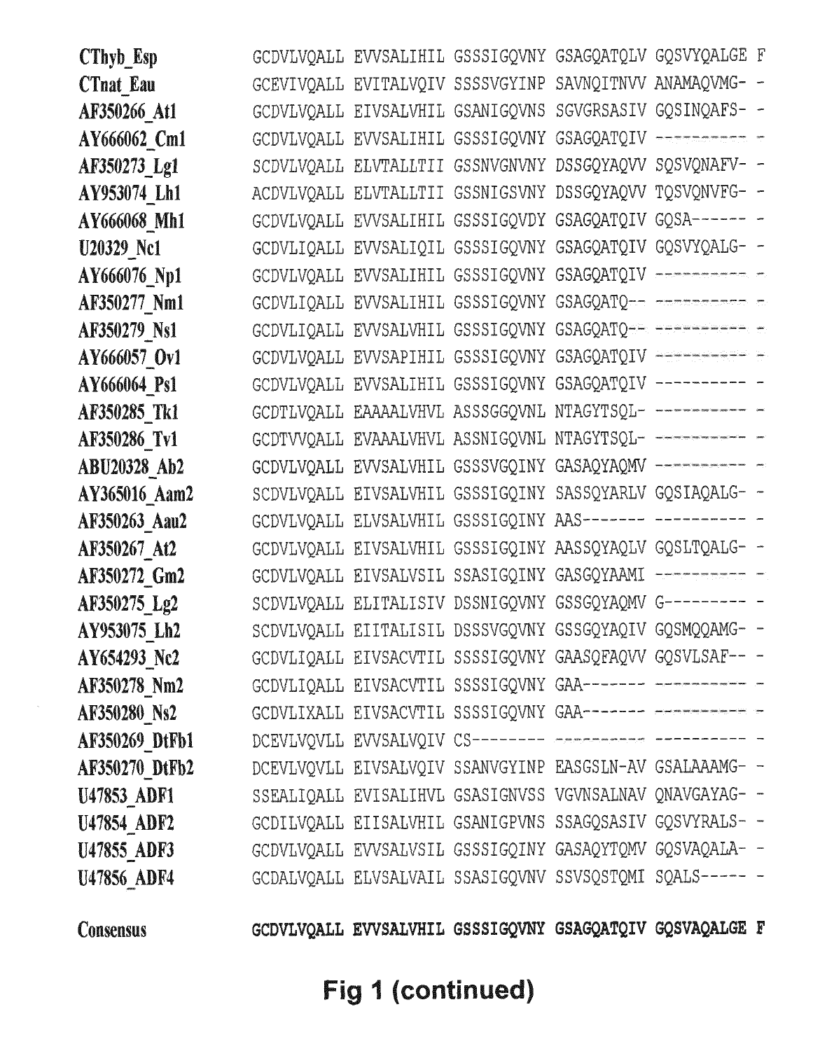

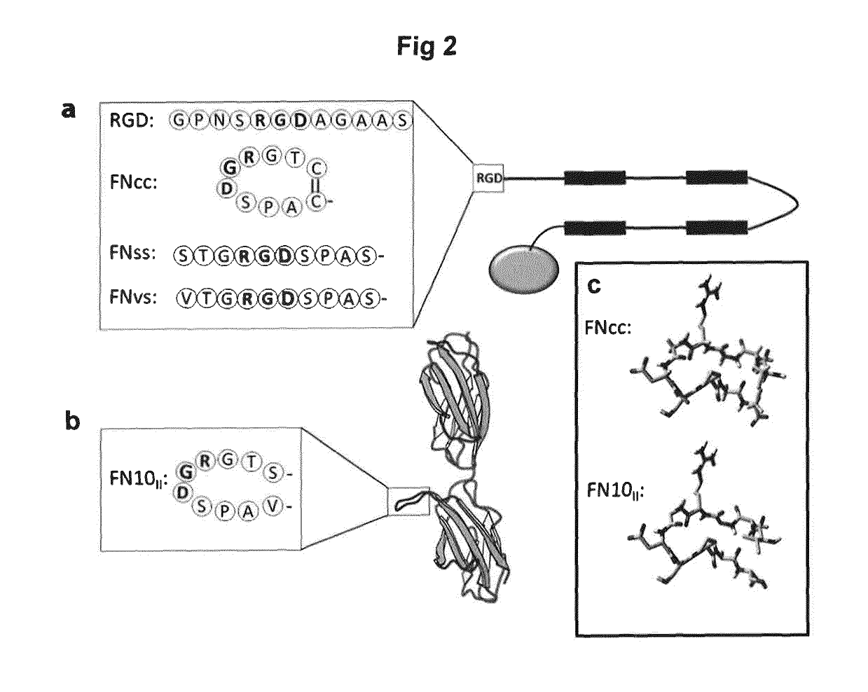

Certain relevant silk constructs with cell binding motifs are illustrated in FIG. 2. FIG. 2a schematically shows the spider silk protein 4RepCT with different RGD motifs genetically introduced to its N-terminus. “RGD” in FIG. 1 a denotes the RGD containing peptide (SEQ ID NO 21) used in Widhe M et al., Biomaterials 34(33): 8223-8234 (2013). “FNᵥₛ” denotes the RGD-containing decapeptide from fibronectin (SEQ ID NO: 22). “FN_{cc}” in FIG. 1 a denotes the same peptide with V and S exchanged to C (SEQ ID NO: 20). “FNₛₛ” denotes the same peptide with V and S exchanged to S (SEQ ID NO: 23). FIG. 1b shows the structure of the 9th and 10th domain of fibronectin, displaying the turn loop containing the RGD motif. FIG. 1c shows a structure model of the RGD loop taken from fibronectin, with the residues V and S mutated to C (adapted from 1FNF.pdb).

In its most general form, FN_{cc} is C¹X¹X²RGDX³X⁴X⁵C² (SEQ ID NO: 9); wherein each of X¹, X², X³, X⁴ and X⁵ are independently selected from natural amino acid residues other than cysteine; and C¹ and C² are connected via a disulphide bond. FN_{cc} is a modified cell-binding motif that imitates the α5β1-specific RGD loop motif of fibronectin by positioning cysteines in precise positions adjacent to the RGD sequence to allow formation of a disulphide-bridge to constrain the chain into a similar type of turn loop. This cyclic RGD cell-binding motif increases the cell adhesion efficacy to a matrix made of a protein containing the cell-binding motif, such as a recombinantly produced spider silk protein. The term “cyclic” as used herein refers to a peptide wherein two amino acid residues are covalently bonded via their side chains, more specifically through a disulfide bond between two cysteine residues. The cyclic RGD cell-binding motif FN_{cc} promotes both proliferation of and migration by primary cells. Human primary cells cultured on a cell scaffold material containing the cyclic RGD cell-binding motif show increased attachment, spreading, stress fiber formation and focal adhesions compared to the same material containing a linear RGD peptide.

In preferred embodiments of FN_{cc}, each of X¹, X², X³, X⁴ and X⁵ are independently selected from the group of amino acid residues consisting of: G, A, V, S, T, D, E, M, P, N and Q. In other preferred embodiments of FN_{cc}, each of X¹ and X³ are independently selected from the group of amino acid residues consisting of: G, S, T, M, N and Q; and each of X², X⁴ and X⁵ are independently selected from the group of amino acid residues consisting of: G, A, V, S, T, P, N and Q. In certain preferred embodiments of FN_{cc}, X¹ is selected from the group of amino acid residues consisting of: G, S, T, N and Q; X³ is selected from the group of amino acid residues consisting of: S, T and Q; and each of X², X⁴ and X⁵ are independently selected from the group of amino acid residues consisting of: G, A, V, S, T, P and N. In some preferred embodiments of FN_{cc}, X¹ is S or T; X² is G, A or V; preferably G or A; more preferably G; X³ is S or T; preferably S; X⁴ is G, A, V or P; preferably G or P; more preferably P; and X⁵ is G, A or V; preferably G or A; more preferably A.

In certain preferred embodiments of FN_{cc}, the cell-binding motif is comprising the amino acid sequence CTGRGDSPAC (SEQ ID NO: 20). Further preferred cyclic RGD cell-binding motifs according to the invention display at least 60%, such as at least 70%, such as at least 80%, such as at least 90% identity to CTGRGDSPAC (SEQ ID NO: 20), with the proviso that position 1 and 10 are always C; position 4 is always R; position 5 is always G; position 6 is always D; and positions 2-3 and 7-9 are never cysteine. It is understood that the non-identical positions among positions 2-3 and 7-9 can be freely selected as set out above.

A preferred group of cell-binding motifs are FN_{cc}, GRKRK, IKVAV, and RGD, and in particular FN_{cc}, such as CTGRGDSPAC.

The spider silk protein is preferably comprising, or consisting of, the protein moieties REP and CT. A preferred spider silk protein has the structure REP-CT. Another preferred spider silk protein has the structure REP-CT. The optional cell-binding motif is arranged either terminally in the spider silk protein, or between the moieties, or within any of the moieties, preferably N-terminally or C-terminally in the spider silk protein.

REP is a repetitive fragment of from 70 to 300 amino acid residues, selected from the group consisting of L(AG)ₙL, L(AG)ₙAL, L(GA)ₙL, and L(GA)ₙGL, wherein
n is an integer from 2 to 10;
each individual A segment is an amino acid sequence of from 8 to 18 amino acid residues, wherein from 0 to 3 of the amino acid residues are not Ala, and the remaining amino acid residues are Ala;
each individual G segment is an amino acid sequence of from 12 to 30 amino acid residues, wherein at least 40% of the amino acid residues are Gly; and
each individual L segment is a linker amino acid sequence of from 0 to 30 amino acid residues; and

CT is a fragment of from 70 to 120 amino acid residues, having at least 70% identity to SEQ ID NO: 3 or SEQ ID NO: 68.

The spider silk protein according to the invention is preferably a recombinant protein, i.e. a protein that is made by expression from a recombinant nucleic acid, i.e. DNA or RNA that is created artificially by combining two or more nucleic acid sequences that would not normally occur together (genetic engineering). The spider silk proteins according to the invention are preferably recombinant proteins, and they are therefore not identical to naturally occurring proteins. In particular, wildtype spidroins are preferably not spider silk proteins according to the invention, because they are not expressed from a recombinant nucleic acid as set out above. The combined nucleic acid sequences encode different proteins, partial proteins or polypeptides with certain functional properties. The resulting recombinant protein is a single protein with functional properties derived from each of the original proteins, partial proteins or polypeptides.

The spider silk protein typically consists of from 140 to 2000 amino acid residues, such as from 140 to 1000 amino acid residues, such as from 140 to 600 amino acid residues, preferably from 140 to 500 amino acid residues, such as from 140 to 400 amino acid residues. The small size is advantageous because longer proteins containing spider silk protein fragments may form amorphous aggregates, which require use of harsh solvents for solubilisation and polymerisation.

The spider silk protein may contain one or more linker peptides, or L segments. The linker peptide(s) may be arranged between any moieties of the spider silk protein, e.g. between the REP and CT moieties, at either terminal end of the spider silk protein or between the spidroin fragment and the cell-binding motif. The linker(s) may provide a spacer between the functional units of the spider silk protein, but may also constitute a handle for identification and purification of the spider silk protein, e.g. a His and/or a Trx tag. If the spider silk protein contains two or more linker peptides for identification and purification of the spider silk protein, it is preferred that they are separated by a spacer sequence, e.g. His₆-spacer-His₆-. The linker may also constitute a signal peptide, such as a signal recognition particle, which directs the spider silk protein to the membrane and/or causes secretion of the spider silk protein from the host cell into the surrounding medium. The spider silk protein may also include a cleavage site in its amino acid sequence, which allows for cleavage and removal of the linker(s) and/or other relevant moieties. Various cleavage sites are known to the person skilled in the art, e.g. cleavage sites for chemical agents, such as CNBr after Met residues and hydroxylamine between Asn-Gly residues, cleavage sites for proteases, such as thrombin or protease 3C, and self-splicing sequences, such as intein self-splicing sequences.

The spidroin fragment and the cell-binding motif are linked directly or indirectly to one another. A direct linkage implies a direct covalent binding between the moieties without intervening sequences, such as linkers. An indirect linkage also implies that the moieties are linked by covalent bonds, but that there are intervening sequences, such as linkers and/or one or more further moieties, e.g. 1-2 NT moieties.

The cell-binding motif may be arranged internally or at either end of the spider silk protein, i.e. C-terminally arranged or N-terminally arranged. It is preferred that the cell-binding motif is arranged at the N-terminal end of the spider silk protein. If the spider silk protein contains one or more linker peptide(s) for identification and purification of the spider silk protein, e.g. a His or Trx tag(s), it is preferred that it is arranged at the N-terminal end of the spider silk protein.

A preferred spider silk protein has the form of an N-terminally arranged cell-bonding motif, coupled by a linker peptide of 0-30 amino acid residues, such as 0-10 amino acid residues, to a REP moiety. Optionally, the spider silk protein has an N-terminal or C-terminal linker peptide, which may contain a purification tag, such as a His tag, and a cleavage site.

The protein moiety REP is fragment with a repetitive character, alternating between alanine-rich stretches and glycine-rich stretches. The REP fragment generally contains more than 70, such as more than 140, and less than 300, preferably less than 240, such as less than 200, amino acid residues, and can itself be divided into several L (linker) segments, A (alanine-rich) segments and G (glycine-rich) segments, as will be explained in more detail below. Typically, said linker segments, which are optional, are located at the REP fragment terminals, while the remaining segments are in turn alanine-rich and glycine-rich. Thus, the REP fragment can generally have either of the following structures, wherein n is an integer:

L(AG)ₙL, such as LA₁G₁A₂G₂A₃G₃A₄G₄A₅G₅L;

L(AG)ₙAL, such as LA₁G₁A₂G₂A₃G₃A₄G₄A₅G₅A₆L;

L(GA)ₙL, such as LG₁A₁G₂A₂G₃A₃G₄A₄G₅A₅L; or

L(GA)ₙGL, such as LG_{I}A_{I}G₂A₂G₃A₃G₄A₄G₅A₅G₆L.

It follows that it is not critical whether an alanine-rich or a glycine-rich segment is adjacent to the N-terminal or C-terminal linker segments. It is preferred that n is an integer from 2 to 10, preferably from 2 to 8, also preferably from 4 to 8, more preferred from 4 to 6, i.e. n=4, n=5 or n=6.

In some embodiments, the alanine content of the REP fragment is above 20%, preferably above 25%, more preferably above 30%, and below 50%, preferably below 40%, more preferably below 35%. It is contemplated that a higher alanine content provides a stiffer and/or stronger and/or less extendible fiber.

In certain embodiments, the REP fragment is void of proline residues, i.e. there are no Pro residues in the REP fragment.

Turning now to the segments that constitute the REP fragment, it is emphasized that each segment is individual, i.e. any two A segments, any two G segments or any two L segments of a specific REP fragment may be identical or may not be identical. Thus, it is not a general feature of the spidroin that each type of segment is identical within a specific REP fragment. Rather, the following disclosure provides the skilled person with guidelines how to design individual segments and gather them into a REP fragment, which is a part of a functional spider silk protein useful in a cell scaffold material.

Each individual A segment is an amino acid sequence having from 8 to 18 amino acid residues. It is preferred that each individual A segment contains from 13 to 15 amino acid residues. It is also possible that a majority, or more than two, of the A segments contain from 13 to 15 amino acid residues, and that a minority, such as one or two, of the A segments contain from 8 to 18 amino acid residues, such as 8-12 or 16-18 amino acid residues. A vast majority of these amino acid residues are alanine residues. More specifically, from 0 to 3 of the amino acid residues are not alanine residues, and the remaining amino acid residues are alanine residues. Thus, all amino acid residues in each individual A segment are alanine residues, with no exception or with the exception of one, two or three amino acid residues, which can be any amino acid. It is preferred that the alanine-replacing amino acid(s) is (are) natural amino acids, preferably individually selected from the group of serine, glutamic acid, cysteine and glycine, more preferably serine. Of course, it is possible that one or more of the A segments are all-alanine segments, while the remaining A segments contain 1-3 non-alanine residues, such as serine, glutamic acid, cysteine or glycine.

In an embodiment, each A segment contains 13-15 amino acid residues, including 10-15 alanine residues and 0-3 non-alanine residues as described above. In a more preferred embodiment, each A segment contains 13-15 amino acid residues, including 12-15 alanine residues and 0-1 non-alanine residues as described above.

It is preferred that each individual A segment has at least 80%, preferably at least 90%, more preferably 95%, most preferably 100% identity to an amino acid sequence selected from the group of amino acid residues 7-19, 43-56, 71-83, 107-120, 135-147, 171-183, 198-211, 235-248, 266-279, 294-306, 330-342, 357-370, 394-406, 421-434, 458-470, 489-502, 517-529, 553-566, 581-594, 618-630, 648-661, 676-688, 712-725, 740-752, 776-789, 804-816, 840-853, 868-880, 904-917, 932-945, 969-981, 999-1013, 1028-1042 and 1060-1073 of SEQ ID NO: 5. Each sequence of this group corresponds to a segment of the naturally occurring sequence of Euprosthenops australis MaSp1 protein, which is deduced from cloning of the corresponding cDNA, see WO2007/078239. Alternatively, each individual A segment has at least 80%, preferably at least 90%, more preferably 95%, most preferably 100% identity to an amino acid sequence selected from the group of amino acid residues 25-36, 55-69, 84-98, 116-129 and 149-158 of SEQ ID NO: 2. Each sequence of this group corresponds to a segment of expressed, non-natural spider silk proteins, which proteins have the capacity to form silk fibers under appropriate conditions. Thus, in certain embodiments of the spidroin, each individual A segment is identical to an amino acid sequence selected from the above-mentioned amino acid segments. Without wishing to be bound by any particular theory, it is envisaged that A segments according to the invention form helical structures or beta sheets.

Furthermore, it has been concluded from experimental data that each individual G segment is an amino acid sequence of from 12 to 30 amino acid residues. It is preferred that each individual G segment consists of from 14 to 23 amino acid residues. At least 40% of the amino acid residues of each G segment are glycine residues. Typically, the glycine content of each individual G segment is in the range of 40-60%.

It is preferred that each individual G segment has at least 80%, preferably at least 90%, more preferably 95%, most preferably 100% identity to an amino acid sequence selected from the group of amino acid residues 20-42, 57-70, 84-106, 121-134, 148-170, 184-197, 212-234, 249-265, 280-293, 307-329, 343-356, 371-393, 407-420, 435-457, 471-488, 503-516, 530-552, 567-580, 595-617, 631-647, 662-675, 689-711, 726-739, 753-775, 790-803, 817-839, 854-867, 881-903, 918-931, 946-968, 982-998, 1014-1027, 1043-1059 and 1074-1092 of SEQ ID NO: 5. Each sequence of this group corresponds to a segment of the naturally occurring sequence of Euprosthenops australis MaSp1 protein, which is deduced from cloning of the corresponding cDNA, see WO2007/078239. Alternatively, each individual G segment has at least 80%, preferably at least 90%, more preferably 95%, most preferably 100% identity to an amino acid sequence selected from the group of amino acid residues 1-24, 37-54, 70-83, 99-115 and 130-148 of SEQ ID NO: 2. Each sequence of this group corresponds to a segment of expressed, non-natural spider silk proteins, which proteins have the capacity to form silk fibers under appropriate conditions. Thus, in certain embodiments of the spidroin in the cell scaffold material, each individual G segment is identical to an amino acid sequence selected from the above-mentioned amino acid segments.

In certain embodiments, the first two amino acid residues of each G segment are not -Gln-Gln-.

There are three subtypes of the G segment. This classification is based upon careful analysis of the Euprosthenops australis MaSp1 protein sequence (see WO2007/078239), and the information has been employed and verified in the construction of novel, non-natural spider silk proteins.

The first subtype of the G segment is represented by the amino acid one letter consensus sequence GQG(G/S)QGG(Q/Y)GG (L/Q)GQGGYGQGA GSS (SEQ ID NO: 6). This first, and generally the longest, G segment subtype typically contains 23 amino acid residues, but may contain as little as 17 amino acid residues, and lacks charged residues or contain one charged residue. Thus, it is preferred that this first G segment subtype contains 17-23 amino acid residues, but it is contemplated that it may contain as few as 12 or as many as 30 amino acid residues. Without wishing to be bound by any particular theory, it is envisaged that this subtype forms coil structures or 3₁-helix structures. Representative G segments of this first subtype are amino acid residues 20-42, 84-106, 148-170, 212-234, 307-329, 371-393, 435-457, 530-552, 595-617, 689-711, 753-775, 817-839, 881-903, 946-968, 1043-1059 and 1074-1092 of SEQ ID NO: 5. In certain embodiments, the first two amino acid residues of each G segment of this first subtype according to the invention are not -Gln-Gln-.

The second subtype of the G segment is represented by the amino acid one letter consensus sequence GQGGQGQG(G/R)Y GQG(A/S)G(S/G)S (SEQ ID NO: 7). This second, generally mid-sized, G segment subtype typically contains 17 amino acid residues and lacks charged residues or contain one charged residue. It is preferred that this second G segment subtype contains 14-20 amino acid residues, but it is contemplated that it may contain as few as 12 or as many as 30 amino acid residues. Without wishing to be bound by any particular theory, it is envisaged that this subtype forms coil structures. Representative G segments of this second subtype are amino acid residues 249-265, 471-488, 631-647 and 982-998 of SEQ ID NO: 5.

The third subtype of the G segment is represented by the amino acid one letter consensus sequence G(R/Q)GQG(G/R)YGQG (A/S/V)GGN (SEQ ID NO: 8). This third G segment subtype typically contains 14 amino acid residues, and is generally the shortest of the G segment subtypes. It is preferred that this third G segment subtype contains 12-17 amino acid residues, but it is contemplated that it may contain as many as 23 amino acid residues. Without wishing to be bound by any particular theory, it is envisaged that this subtype forms turn structures. Representative G segments of this third subtype are amino acid residues 57-70, 121-134, 184-197, 280-293, 343-356, 407-420, 503-516, 567-580, 662-675, 726-739, 790-803, 854-867, 918-931, 1014-1027 of SEQ ID NO: 5.

Thus, in preferred embodiments of the spidroin in the cell scaffold material, each individual G segment has at least 80%, preferably 90%, more preferably 95%, identity to an amino acid sequence selected from SEQ ID NO: 6, SEQ ID NO: 7 and SEQ ID NO: 8.

In an embodiment of the alternating sequence of A and G segments of the REP fragment, every second G segment is of the first subtype, while the remaining G segments are of the third subtype, e.g. . . . A₁GₛₕₒᵣₜA₂G_{long}A₃GₛₕₒᵣₜA₄G_{long}A₅Gₛₕₒᵣₜ . . . In another embodiment of the REP fragment, one G segment of the second subtype interrupts the G segment regularity via an insertion, e.g. . . . A₁GₛₕₒᵣₜA₂G_{long}A₃G_{mid}A₄GₛₕₒᵣₜA₅G_{long} . . .

Each individual L segment represents an optional linker amino acid sequence, which may contain from 0 to 30 amino acid residues, such as from 0 to 20 amino acid residues. While this segment is optional and not critical for the function of the spider silk protein, its presence still allows for fully functional spider silk proteins and polymers thereof which form fibers, films, foams and other structures. There are also linker amino acid sequences present in the repetitive part (SEQ ID NO: 5) of the deduced amino acid sequence of the MaSp1 protein from Euprosthenops australis. In particular, the amino acid sequence of a linker segment may resemble any of the described A or G segments, but usually not sufficiently to meet their criteria as defined herein.

As shown in WO2007/078239, a linker segment arranged at the C-terminal part of the REP fragment can be represented by the amino acid one letter consensus sequences ASASAAASAA STVANSVS (SEQ ID NO: 32) and ASAASAAA (SEQ ID NO: 33), which are rich in alanine. In fact, the second sequence can be considered to be an A segment according to the definition herein, whereas the first sequence has a high degree of similarity to A segments according to this definition. Another example of a linker segment has the one letter amino acid sequence GSAMGQGS (SEQ ID NO: 34), which is rich in glycine and has a high degree of similarity to G segments according to the definition herein. Another example of a linker segment is SASAG (SEQ ID NO: 35).

Representative L segments are amino acid residues 1-6 and 1093-1110 of SEQ ID NO: 5; and amino acid residues 159-165 of SEQ ID NO: 2, but the skilled person will readily recognize that there are many suitable alternative amino acid sequences for these segments. In one embodiment of the REP fragment, one of the L segments contains 0 amino acids, i.e. one of the L segments is void. In another embodiment of the REP fragment, both L segments contain 0 amino acids, i.e. both L segments are void. Thus, these embodiments of the REP fragments according to the invention may be schematically represented as follows: (AG)ₙL, (AG)ₙAL, (GA)ₙL, (GA)ₙGL; L(AG)ₙ, L(AG)ₙA, L(GA)ₙ, L(GA)ₙG; and (AG)ₙ, (AG)ₙA, (GA)ₙ, (GA)ₙG. Any of these REP fragments are suitable for use with any CT fragment as defined below.

The CT fragment of the spidroin in the cell scaffold material has a high degree of similarity to the C-terminal amino acid sequence of spider silk proteins. As shown in WO2007/078239, this amino acid sequence is well conserved among various species and spider silk proteins, including MaSp1, MaSp2 and MiSp (minor ampullate spidroin). A consensus sequence of the C-terminal regions of MaSp1 and MaSp2 is provided as SEQ ID NO: 4. In FIG. 1, the MaSp proteins (SEQ ID NO: 36-66) presented in Table 1 are aligned, denoted with GenBank accession entries where applicable:

TABLE 1
Spidroin CT fragments

| Species and spidroin | Entry |
|---|---|
| Euprosthenops sp MaSp1 (Pouchkina-Stantcheva*) | Cthyb_Esp |
| Euprosthenops australis MaSp1 (SEQ ID NO: 3) | CTnat_Eau |
| Argiope trifasciata MaSp1 | AF350266_At1 |
| Cyrtophora moluccensis Sp1 | AY666062_Cm1 |
| Latrodectus geometricus MaSp1 | AF350273_Lg1 |
| Latrodectus hesperus MaSp1 | AY953074_Lh1 |
| Macrothele holsti Sp1 | AY666068_Mh1 |
| Nephila clavipes MaSp1 | U20329_Nc1 |
| Nephila pilipes MaSp1 | AY666076_Np1 |
| Nephila madagascariensis MaSp1 | AF350277_Nm1 |
| Nephila senegalensis MaSp1 | AF350279_Ns1 |
| Octonoba varians Sp1 | AY666057_Ov1 |
| Psechrus sinensis Sp1 | AY666064_Ps1 |
| Tetragnatha kauaiensis MaSp1 | AF350285_Tk1 |
| Tetragnatha versicolor MaSp1 | AF350286_Tv1 |
| Araneus bicentenarius Sp2 | ABU20328_Ab2 |
| Argiope amoena MaSp2 | AY365016_Aam2 |
| Argiope aurantia MaSp2 | AF350263_Aau2 |
| Argiope trifasciata MaSp2 | AF350267_At2 |
| Gasteracantha mammosa MaSp2 | AF350272_Gm2 |
| Latrodectus geometricus MaSp2 | AF350275_Lg2 |
| Latrodectus hesperus MaSp2 | AY953075_Lh2 |
| Nephila clavipes MaSp2 | AY654293_Nc2 |
| Nephila madagascariensis MaSp2 | AF350278_Nm2 |
| Nephila senegalensis MaSp2 | AF350280_Ns2 |
| Dolomedes tenebrosus Fb1 | AF350269_DtFb1 |
| Dolomedes tenebrosus Fb2 | AF350270_DtFb2 |
| Araneus diadematus ADF-1 | U47853_ADF1 |
| Araneus diadematus ADF-2 | U47854_ADF2 |
| Araneus diadematus ADF-3 | U47855_ADF3 |
| Araneus diadematus ADF-4 | U47856_ADF4 |

*Comparative Biochemistry and Physiology, Part B 138: 371-376 (2004)

It is not critical which specific CT fragment is present in the spider silk protein in the cell scaffold material. Thus, the CT fragment can be selected from any of the amino acid sequences shown in FIG. 1 and Table 1 or sequences with a high degree of similarity, such as the MiSp CT fragment SEQ ID NO: 68 from Araneus ventricosus (Genbank entry AFV 31615). A wide variety of C-terminal sequences can be used in the spider silk protein.

The sequence of the CT fragment has at least 50% identity, preferably at least 60%, more preferably at least 65% identity, or even at least 70% identity, to the consensus amino acid sequence SEQ ID NO: 4, which is based on the amino acid sequences of FIG. 1.

A representative CT fragment is the Euprosthenops australis sequence SEQ ID NO: 3 or amino acid residues 180-277 of SEQ ID NO: 27. Another representative CT fragment is the MiSp sequence SEQ ID NO: 68. Thus, in one embodiment, the CT fragment has at least 70%, such as at least 80%, such as at least 85%, preferably at least 90%, such as at least 95%, identity to SEQ ID NO: 3, amino acid residues 180-277 of SEQ ID NO: 27, or any individual amino acid sequence of FIG. 1 and Table 1, or SEQ ID NO: 68. For example, the CT fragment may be identical to SEQ ID NO: 3, amino acid residues 180-277 of SEQ ID NO: 27, or any individual amino acid sequence of FIG. 1 and Table 1, or SEQ ID NO: 68.

The CT fragment typically consists of from 70 to 120 amino acid residues. It is preferred that the CT fragment contains at least 70, or more than 80, preferably more than 90, amino acid residues. It is also preferred that the CT fragment contains at most 120, or less than 110 amino acid residues. A typical CT fragment contains approximately 100 amino acid residues.

The term “% identity”, as used herein, is calculated as follows. The query sequence is aligned to the target sequence using the CLUSTAL W algorithm (Thompson et al, Nucleic Acids Research, 22:4673-4680 (1994)). A comparison is made over the window corresponding to the shortest of the aligned sequences. The amino acid residues at each position are compared, and the percentage of positions in the query sequence that have identical correspondences in the target sequence is reported as % identity.

The term “% similarity”, as used herein, is calculated as described above for “% identity”, with the exception that the hydrophobic residues Ala, Val, Phe, Pro, Leu, Ile, Trp, Met and Cys are similar; the basic residues Lys, Arg and His are similar; the acidic residues Glu and Asp are similar; and the hydrophilic, uncharged residues Gln, Asn, Ser, Thr and Tyr are similar. The remaining natural amino acid Gly is not similar to any other amino acid in this context.

Throughout this description, alternative embodiments according to the invention fulfill, instead of the specified percentage of identity, the corresponding percentage of similarity. Other alternative embodiments fulfill the specified percentage of identity as well as another, higher percentage of similarity, selected from the group of preferred percentages of identity for each sequence. For example, a sequence may be 70% similar to another sequence; or it may be 70% identical to another sequence; or it may be 70% identical and 90% similar to another sequence.

In a preferred spider silk protein according to the invention, the REP-CT fragment has at least 70%, such as at least 80%, such as at least 85%, preferably at least 90%, such as at least 95%, identity to SEQ ID NO: 2 or to amino acid residues 18-277 of SEQ ID NO: 27 or to amino acid residues 18-272 of SEQ ID NO: 69.

In one preferred spider silk protein according to the invention, the protein has at least 70%, such as at least 80%, such as at least 85%, preferably at least 90%, such as at least 95%, identity to SEQ ID NO: 25, 27 or 69. In a particularly preferred embodiment, the spider silk protein according to the invention is SEQ ID NO: 25, 27 or 69.

The cell scaffold material according to the invention preferably comprises a protein or peptide according to the invention displaying the cyclic RGD cell-binding motif. The cyclic RGD cell-binding motif may be exposed from short synthetic peptides or longer synthetic or recombinant proteins, which may in turn be attached to or associated with a matrix or support.

The cell scaffold material preferably comprises a protein polymer, which protein polymer in turn is containing the silk protein according to the invention as a repeating structural unit, i.e. the protein polymer contains or consists of a polymer of the silk protein according to the invention. This implies that the protein polymer contains or consists of an ordered plurality of silk proteins according to the invention, typically well above 100 silk protein units, e.g. 1000 silk protein units or more. In a preferred embodiment, the cell scaffold material according to the invention consists of the protein polymer.

The magnitude of silk protein units in the polymer implies that the protein polymer obtains a significant size. In a preferred embodiment, the protein polymer has a size of at least 0.01 μm in at least two dimensions. Thus, the term “protein polymer” as used herein relates to silk protein polymers having a thickness of at least 0.01 μm, such as at least 0.1 μm, preferably macroscopic polymers that are visible to the human eye, i.e. having a thickness of at least 1 μm, such as up 10 μm. The term “protein polymer” does not encompass unstructured aggregates or precipitates. While monomers/dimers of the spider silk protein are water soluble, it is understood that the protein polymers according to the invention are solid structures, i.e. not soluble in water. The protein polymers are comprising monomers of the silk proteins according to the invention as a repeating structural unit.

The protein polymer according to the invention is typically provided in a physical form selected from the group consisting of fiber, film, coating, foam, net, fiber-mesh, sphere and capsule. According to one embodiment, it is preferable that the protein polymer according to the invention is a fiber, film or fiber-mesh. According to certain embodiments, it is preferable that the protein polymer has a three-dimensional form, such as a foam or a fiber-mesh. One preferred embodiment involves thin (typically 0.01-0.1 μm thickness) coatings made of the protein polymer, which are useful for coating of stents and other medical devices. The term “foam” is comprising a porous foam with channels connecting the bubbles of the foam, sometimes to the extent that it can even be regarded as a three-dimensional net or mesh of fibers.

In a preferred embodiment, the protein polymer is in a physical form of a free-standing matrix, such as a free-standing film. This is highly useful as it allows for transfer of a cell sheet where needed, e.g. in an in vivo situation where cells need to be transferred as a cell sheet to e.g. a wound area.

The fiber, film or fiber-mesh typically has a thickness of at least 0.1 μm, preferably at least 1 μm. It is preferred that the fiber, film or fiber-mesh has a thickness in the range of 1-400 μm, preferably 60-120 μm. It is preferred that fibers have a length in the range of 0.5-300 cm, preferably 1-100 cm. Other preferred ranges are 0.5-30 cm and 1-20 cm. The fiber has the capacity to remain intact during physical manipulation, i.e. can be used for spinning, weaving, twisting, crocheting and similar procedures. The film is advantageous in that it is coherent and adheres to solid structures, e.g. the plastics in microtiter plates. This property of the film facilitates washing and regeneration procedures and is very useful for separation purposes.

The spider silk protein according to the invention harbors an internal solid support activity in the REP-CT moieties, and optionally also a desired cell-binding activity in the cell-binding motif, and these activities are employed in the cell scaffold material. The cell scaffold material provides a high and predictable density of the selective interaction activity towards an organic target. Losses of valuable protein moieties with selective interaction activity are minimized, since all expressed protein moieties are associated with the cell scaffold material.

The polymers which are formed from the silk proteins according to the invention are solid structures and are useful for their physical properties, especially the useful combination of high strength, elasticity and light weight. A particularly useful feature is that the REP-CT moieties of the spider silk protein are biochemically robust and suitable for regeneration, e.g. with acid, base or chaotropic agents, and suitable for heat sterilization, e.g. autoclaving at 120° C. for 20 min. The polymers are also useful for their ability to support cell adherence and growth.

The properties derived from the REP-CT moieties are attractive in development of new materials for medical or technical purposes. In particular, the cell scaffold materials according to the invention are useful as scaffolds for cell immobilization, cell culture, cell differentiation, tissue engineering and guided cell regeneration. They are also useful in preparative and analytical separation procedures, such as chromatography, cell capture, selection and culture, active filters, and diagnostics. The cell scaffold materials according to the invention are also useful as in medical devices, such as implants and stents, e.g. as coatings.

In a preferred embodiment, the cell scaffold material comprises a protein polymer, which is consisting of a silk protein according to the invention as a repeating structural unit. And in a further preferred embodiment, the cell scaffold material is a protein polymer, which is consisting of a silk protein according to the invention as a repeating structural unit. The silk protein is a fibroin or a spider silk protein.

In the second step, an aqueous mixture of a sample of the eukaryotic cells with the silk protein is prepared. This can preferably be achieved by mixing the aqueous solution from the previous step with a liquid cell suspension or by dispersing a cell pellet. The liquid component of the aqueous mixture should be suitable for the respective eukaryotic cell in terms of buffering capacity, ion strength and pH. Suitable media for cell culture and cell handling are well-known in the art e.g. DMEM, Ham's Nutrient Mixtures, Minimal Essential Medium Eagle, and RPMI.

It is preferred that the eukaryotic cells are mammalian cells, and preferably human cells, including primary cells, cell lines and stem cells. Useful examples of primary cells and cell lines include endothelical cells, fibroblasts, keratinocytes, skeletal muscle satellite cells, skeletal muscle myoblasts, Schwann cells, pancreatic β-cells, pancreatic islet cells, hepatocytes and glioma-forming cells. The stem cells are preferably human pluripotent stem cells (hPSCs), such as embryonic stem cells (ESC) and induced pluripotent cells (iPS). Useful examples of stem cells include mesenchymal stem cells. The cells may also preferably be a combination of at least two different mammalian cell types, such as those set out above.

In the second step, it is critical that silk protein remains dissolved in the aqueous mixture. By the term “dissolved” means that the cells are added to the silk protein before the silk assembly process has been developed, when the silk proteins predominantly form bonds with the surrounding water molecules. When the silk assembly process has been developed, irreversible formation of ordered polymers with predominantly intra- and intermolecular bonds between the silk proteins occurs. It is understood that the polymerization is a continuous process, but according to the present invention, the cells should be added to the dissolved silk protein as early as possible in view of the desired final format of the final macrostructure. It is preferred that the cells are added when at least some, and preferably most of or even substantially all of the silk proteins remain dissolved. Thus for instance, if the desired format is a foam, the cells should be added before foaming or to the wet foam when it is newly made by introduction of air into the liquid, and not when the foam has polymerized into a silk macrostructure.

Optionally, the aqueous mixture may contain further components which are desirable to integrate in the macrostructure. For instance, the aqueous mixture may contain cell-binding proteins and polypeptides, such as laminins.

In the third step, the silk protein is allowed to assemble into a water-insoluble macrostructure in the presence of the eukaryotic cells. Proteins structures according to the invention are assembled spontaneously from the silk proteins according to the invention under suitable conditions, and the assembly into polymers is promoted by the presence of shearing forces and/or an interface between two different phases e.g. between a solid and a liquid phase, between air and a liquid phase or at a hydrophobic/hydrophilic interface, e.g. a mineral oil-water interface. The presence of the resulting interface stimulates polymerization at the interface or in the region surrounding the interface, which region extends into the liquid medium, such that said polymerizing initiates at said interface or in said interface region. Various protein structures can be produced by adapting the conditions during the assembly. For instance, if the assembly is allowed to occur in a container that is gently wagged from side to side, a fiber is formed at the air-water interface. If the mixture is allowed to stand still, a film is formed at the air-water interface. If the mixture is evaporated, a film is formed at the bottom of the container. If oil is added on top of the aqueous mixture, a film is formed at the oil-water interface, either if allowed to stand still or if wagged. If the mixture is foamed, e.g. by bubbling of air or whipping, the foam is stable and solidifies with time. The new macrostructure may be allowed to form in any suitable cell culture well. Optionally, the culture well surface is pre-coated with a silk macrostructure or with other substances, e.g. gelatin.

The assembly into water-insoluble macrostructure results in formation of a scaffold material for cultivating the eukaryotic cells. Thus, the very cells to be cultured are present already during assembly of the scaffold material and become integrated within the cell material. Thereby, the cells become surrounded by and embedded in the spider silk macrostructure. This has advantageous effect in terms of viability, proliferative capacity, cell spreading and attachment in the subsequent cell culture. Furthermore, the co-presence of the cells in the assembly of the macrostructure achieves formation of cavities and pores in the scaffold material which would otherwise not have existed.

In the fourth step, the eukaryotic cells are maintained within the scaffold material under conditions suitable for cell culture, which are well known to the skilled person and exemplified herein. This advantageously allows for the cells to grow integrated with the scaffold material. This means that the cells are not just growing attached to the very surface of the scaffold material, but also within cavities and pores in the scaffold material which have been formed due to air bubbles and the co-presence of the cells in the assembly of the macrostructure.

According to a second aspect, the present invention provides a process for manufacturing a cell culture product comprising (i) a scaffold material for cultivating eukaryotic cells; and (ii) eukaryotic cells, which are growing integrated with the scaffold material. The method is preferably carried out in vitro. The method is comprising the steps:
(a) providing an aqueous solution of a silk protein capable of assembling into a water-insoluble macrostructure, wherein the silk protein optionally contains a cell-binding motif;
(b) preparing an aqueous mixture of a sample of the eukaryotic cells with the silk protein, wherein the silk protein remains dissolved in the aqueous mixture; and
(c) allowing the silk protein to assemble into a water-insoluble macrostructure in the presence of the eukaryotic cells, thereby forming the scaffold material for cultivating the eukaryotic cells.

Preferred embodiments and variants of the manufacturing process are evident from the above disclosure of the method for the cultivation of eukaryotic cells which is including corresponding steps.

According to a third aspect, the present invention provides a cell culture product comprising (i) a scaffold material for cultivating eukaryotic cells, which is a water-insoluble macrostructure of a silk protein capable of assembling into a water-insoluble macrostructure, wherein the silk protein optionally contains a cell-binding motif; and (ii) eukaryotic cells, which are growing integrated with the scaffold material.

This means that the cells are not just growing attached to the very surface of the scaffold material, but also within cavities and pores in the scaffold material which have been formed e.g. due to the co-presence of the cells in the assembly of the macrostructure.

Preferred embodiments and variants of the cell culture product are evident from the above disclosure of the method for the cultivation of eukaryotic cells which is including corresponding features.

In a preferred embodiment, the cell culture product according to the invention is obtainable or obtained by the manufacturing process according to the invention. The co-presence of the cells in the assembly of the macrostructure achieves formation of cavities and pores in the scaffold material which would otherwise not have existed.

According to a fourth and final aspect, the present invention provides a novel use of a silk protein capable of assembling into a water-insoluble macrostructure in the formation of a scaffold material for cultivating eukaryotic cells in the presence of said cells; wherein the scaffold material is a water-insoluble macrostructure of the silk protein; and wherein the silk protein optionally contains a cell-binding motif. The use is preferably carried out in vitro.

Preferred embodiments and variants of the use are evident from the above disclosure of the method for the cultivation of eukaryotic cells which is including corresponding features.

In summary, novel methods for formulation of cell-containing silk scaffolds have been developed, where the cells are added to the silk protein before the silk assembly process has been developed. The following Examples demonstrate how the cells are affected by incorporation into the silk scaffolds, in terms of viability, proliferative capacity, cell spreading and attachment. To survey the generality of the method, a broad repertoire of mammalian cells, ranging from stable cell lines to primary cells, of both mouse and human origin (Table 2) has been analyzed. Maintenance of specific cell functions for certain cell types, such as production of extracellular matrix components, differentiation and responsiveness to glucose stimulation, has also been confirmed.

TABLE 2
Tested mammalian cells

| Silk | | Cell | Cell | | Cell |
|---|---|---|---|---|---|
| format | Motif | type* | viability** | Proliferation*** | integration |
| Fiber:air | FN_{CC} | HSkMC | +++ | +++ | Yes, |
| | | | | | cryosections |
| | | | | | and confocal |
| Fiber:oil | FN_{CC} | HSkMC | +++ | +++ | Yes, |
| | | | | | cryosections |
| Fiber:air | FN_{CC} | HSkMC:HDMEC | +++ | ++ | Yes, |
| | | | | | Cryosections |
| Fiber:air | FN_{CC} | HDMEC | +++ | +++ | Yes, |
| | | | | | cryosections |
| Fiber:oil | GRKRK | HSkMC | + | ++ | Yes, |
| | | | | | cryosections |
| Fiber:oil | GRKRK | HSkMC:HDMEC | + | ++ | Yes, |
| | | | | | cryosections |
| Fiber:air | FN_{CC} | HSMM | +++ | ++ | Yes, |
| | | | | | cryosections |
| Fiber:air | FN_{CC} | HSMM:HDMEC | +++ | ++ | Yes, |
| | | | | | cryosections |
| Fiber:air | FN_{CC} | HDFn:HDMEC | +++ | +++ | Yes, |
| | | | | | cryosections |
| Fiber:air | FN_{CC} | HDFn | | +++ | Yes, |
| | | | | | cryosections |
| Fiber:oil | FN_{CC} | HDFn | | | Yes, |
| | | | | | cryosections |
| Foam | FN_{CC} | HDFn | | ++ | |
| Fiber:oil | FN_{CC} | HaCaT | | | Yes, |
| | | | | | cryosections |
| Fiber:air | FN_{CC} | HaCaT | | | Yes, |
| | | | | | cryosections |
| Foam | FN_{CC} | HaCaT | +++ | ++ | |
| Foam | FN_{CC} | mEC | +++ | ++ | Yes, |
| | | | | | cryosections |
| Fiber:air | FN_{CC} | mEC | +++ | ++ | |
| Foam | FN_{CC} | mMSC | +++ | ++ | Yes, |
| | | | | | cryosections, |
| | | | | | confokal |
| Fiber:air | FN_{CC} | mMSC | +++ | ++ | Yes, |
| | | | | | cryosections |
| Fiber:oil | FN_{CC} | mMSC | ++ | ++ | Yes, |
| | | | | | cryosections |
| Foam | FN_{CC} | hMSC | +++ | ++ | |
| Fiber:air | FN_{CC} | hMSC | +++ | ++ | |
| Foam | FN_{CC} | Schwann | +++ | ++ | |
| | | cells | | | |
| Foam | IKVAV | Schwann | +++ | ++ | |
| | | cells | | | |
| Foam | FN_{CC}:IKVAV | Schwann | +++ | ++ | |
| | | cells | | | |
| Fiber:air | FN_{CC} | Schwann | +++ | ++ | |
| | | cells | | | |
| Fiber:air | IKVAV | Schwann | +++ | ++ | |
| | | cells | | | |
| Fiber:air | FN_{CC}:IKVAV | Schwann | +++ | ++ | |
| | | cells | | | |
| Foam | FN_{CC} | Min6m9 | ++ | ++ | Yes, |
| | | | | | cryosections |
| Foam | 2R | Min6m9 | + | ++ | |
| Foam | FN:2R | Min6m9 | + | ++ | |
| Foam | FN:2R | MIP | +++ | ++ | |
| Foam | FN | Human | | + | |
| | | islets | | | |
| Foam | FN:2R | Human | | + | |
| | | islet cells | | | |
| Foam | FN | hESC | ++ | ++ | |
| Film | FN | hESC | ++ | ++ | |
| Foam | FN | hIPS | ++ | ++ | |
| Film | FN | hIPS | ++ | ++ | |
| Film | FN | SMC | ++ | ++ | |
| Film | FN | HUVEC | ++ | ++ | |
| Foam | FN | HUVEC | ++ | ++ | |

*HSkMC = Human skeletal muscle satellite cells; HDMEC = Human Dermal Microvascular Endothelial cells; HSMM = human skeletal muscle myoblasts; HDFn = Human dermal fibroblasts; HaCaT = human keratinocyte cell line; mEC = mouse Endothelial cells; mMSC = mouse Mesenchymal stem cells; hMSC = human Mesenchymal stem cells; Min6m9 = Pancreatic β-cell line; MIP = Islets from MIP-GFP mice; hESC = human embryonal stem cells; hIPS = human induced pluripotent stem cells; SMC = smooth muscle cells; HUVEC = Human umbilical vein endothelial cells
**+ = 50-70%; ++ =70-90%; +++ = >90%
***+ = cells increase day 1-7; ++ = cells increase day 1-14; +++ = cells increase day 1-21

### EXAMPLES

### Example 1

### Materials and Methods

### Recombinant Spider Silk Protein Preparation

Production of recombinant silk proteins in in E. coli and the following purification were done essentially as described in Hedhammar M et al., Biochemistry 47(11):3407-3417 (2008) and Hedhammar M et al., Biomacromolecules 11: 953-959 (2010).

Briefly, Escherichia coli BL21(DE3) cells (Merck Biosciences) with the expression vector for the target protein were grown at 30° C. in Luria-Bertani medium containing kanamycin to an OD₆₀₀ of 0.8-1 and then induced with isopropyl β-D-thiogalactopyranoside and further incubated for at least 2 h. Thereafter, cells were harvested and resuspended in 20 mM Tris-HCl (pH 8.0) supplemented with lysozyme and DNase I. After complete lysis, the supernatants from centrifugation at 15,000 g were loaded onto a column packed with Ni Sepharose (GE Healthcare, Uppsala, Sweden). The column was washed extensively before elution of bound proteins with 300 mM imidazole. Fractions containing the target proteins were pooled and dialyzed against 20 mM Tris-HCl (pH 8.0). The target protein was released from the tags by proteolytic cleavage. To remove the released HisTrxHis tag, the cleavage mixture was loaded onto a second Ni Sepharose column and the flowthrough was collected. The protein content was determined from the absorbance at 280 nm.

The protein solutions obtained were purified from lipopolysaccharides (Ips) as described in Hedhammar et al., Biomacromolecules 11:953-959 (2010). The protein solutions were sterile filtered (0.22 μm) before being used to prepare scaffolds (film, foam, coatings or fibers).

The recombinant spider silk proteins were successfully expressed in E coli and purified with similar yield and purity as the original 4RepCT.

The partial spider silk protein 4RepCT (SEQ ID NO: 2) was used as base for all proteins used. A functionalized version of 4RepCT with the modified cell binding motif from fibronectin, denoted FNcc-4RepCT in the experimental section (SEQ ID NO: 27), was used for most of the experiments. Other versions, 2RepRGD2RepCT (“2R”, SEQ ID NO: 28) and 3RepRGD1RepCT (“3R”, SEQ ID NO: 29), with the RGD peptide inserted within the repetitive part, was used for some of the experiments with endocrine cells and other cells. Another version GRKRK-4RepCT (SEQ ID NO: 30), with the GRKRK peptide inserted at the N-terminus, was used for some of the experiments with muscle satellite cells. Another version, IKVAV-4RepCT (SEQ ID NO: 31) with the IKVAV peptide inserted at the N-terminus, was used for some of the experiments with Schwann cells.

### Cell Culture

### Mesenchymal Stem Cells (MSC)

Mouse Mesenchymal stem cells (mMSC, Gibco) at a passage of 8-14 were cultured in DMEM F12 HAM supplemented with 10% Fetal Bovine Serum (Mesenchymal Stem Cell Qualified, USDA Approved Regions, Gibco).

Human Mesenchymal stem cells (hMSC) at a passage of 8 (Gibco) from bone marrow were grown in culture flasks coated with CELLstart (Gibco) in complete StemPro MSC serum free medium CTS (Gibco) containing 2 mM Glutamax.

### Endothelial Cells (EC)

Mouse Endothelial cells (Cell Biologics) were cultured at a passage of 7-9 in complete endothelial cell media MV (PromoCell GmbH, Germany).

Human Dermal Microvascular Endothelial cells (HDMEC) (PromoCell GmbH, Germany) isolated from dermis from adult donors were cultured culture flasks coated with gelatin (Sigma Aldrich) in complete endothelial cell media MV (PromoCell GmbH, Germany).

### Fibroblasts (HDFn)

Human dermal fibroblasts, HDF (ECACC, Salisbury, UK) were used in passage 8-11. Culture medium, DMEM F12 ham supplemented with 5% FBS (Sigma), was changed every 2nd-3rd day.

### Keratinocytes (HaCaT)

HaCaT (human keratinocyte cell line, spontaneously transformed), were cultured in DMEM F12 ham supplemented with 5% FBS (Sigma). Medium was changed every 2nd-3rd day.

### Human Skeletal Muscle Satellite Cells (Hsk)

Human skeletal muscle satellite cells, HskMSC (ScienCell Research Laboratories, Carlsbad, Calif.) and human skeletal muscle myoblasts (HSMM, Lonza, Belgium) were used from passage 2-6. Skeletal muscle culture medium, SkMCM (ScienCell Research Laboratories), with skeletal muscle cell growth supplement, SkMCGS (ScienCell Research Laboratories) or SkGM-2 BulletKit (for HSMM, Lonza) and 5% FBS (ScienCell Research Laboratories or Lonza, respectively) was changed every second day.

### Schwann Cells

Schwann cells (3H Biomedical, Uppsala, Sweden) at a passage of 2-6 were cultured in Schwann cell medium (SCM, 3H Biomedical) supplemented with 5% FBS and Schwann cell growth supplement (SCGS, 3H Biomedical) and penicillin/streptomycin solution (3H Biomedical).

### Endocrine Cells

Pancreatic β-cell line MIN6m9 at passage 27-35 were cultured in DMEM (Gibco) supplemented with β-mercaptoethanol (50 μM), penicillin (100 U mL⁻¹), streptomycin (100 ug mL⁻¹), 10% heat-inactivated FBS and glucose (11 mM).

Islets from MIP-GFP mice, all inbred in the animal core facility at Karolinska Institutet, were isolated from pancreas by injecting 1.2 mg/ml collagenase into the bile duct. The pancreas was thoroughly taken out and put into a flask containing collagenase with same concentration as above. The flask was then put into a 37° C. water bath for 15 min. There after the islets were washed and handpicked under a stereomicroscope. To disperse the islets into cells, the islets were first washed two times in PBS without Ca²⁺ and Mg²⁺ and incubated in Accutase (Gibco) for 5 min at 37° C. The cells were counted and cultured in RPMI 1640 medium (Gibco) supplemented with L-glutamine (2 mM), penicillin (100 U mL⁻¹), streptomycin (100 ug mL⁻¹) and 10% heat-inactivated fetal bovine serum (FBS).

Human islets were obtained from the unavoidable excess of islets generated within the Nordic Network for Clinical Islet Transplantation. Only organ donors who explicitly had agreed to donate for scientific purposes were included. Informed written consent to donate organs for medical and research purposes was obtained from donors, or relatives of donors, by the National Board of Health and Welfare (Socialstyrelsen), Sweden. Experimental procedures were done according to the approved ethical permit from the Ethical Committee for Human Research (permit number 2011/14667-32). The human cells were cultured in CMRL-1066 (ICN Biomedicals) supplemented with HEPES (10 mM), L-glutamine (2 mM), Gentamycin (50 mg ml⁻¹), Fungizone (0.25 mg ml⁻¹, Gibco), Ciprofloxacin (20 mg ml⁻¹, Bayer Healthcare AG), nicotinamide (10 mM), and 10% heat inactivated FBS.

### Hepatocytes

Rodent Hepatocytes (liver cells) are isolated by enzymatic (1,2 mg/ml Collagenase P in pH 7.4 HBSS buffer supplemented with 25 mM Hepes, 0,25% w/v BSA) collagenase treatment of the liver, digested by a continuous mechanic shaking in 37° C. for 20 minutes, separated and cultured in RPMI-1640 medium supplemented with 10% FBS (Invitrogen).

### Glioma Forming Cell Line

Glioma forming cell line GL261 is cultured in 10% FBS containing DMEM (Invitrogen) with medium change every 2-3rd day.

### Co-Cultures

Hsk cells in co-culture with EC were cultured in SkMCM culture media. Endocrine cells in co-culture with MSC and EC cultured in RPMI 1640 medium (Gibco), StemPro MSC serum free medium CTS (Gibco) containing 2 mM Glutamax and endothelial cell media MV (PromoCell GmbH, Germany) at a ratio of 50:25:25.

Formulation of Silk Scaffolds with Integrated Cells

### Fiber Formation

Silk protein (0.5-3 mg) was mixed with 0.5-2 million cells in respective culture media in a total volume of 2-4 ml. The fiber formation together with cells was performed in RT under gentle wagging for 1-3 hours. The formed fibers were then washed in 1×PBS and thereafter transferred into non-tissue treated 12 or 24-well plates and further kept in culture by adding fresh media (0.5mL in 24-well plate or 1mL in 12-well plate).

For fiber formation against oil, 3-4 ml of either FC40 (3M), HFE7100 or HFE7500 (Novec) oils were used.

For pre-made fibers, 70 000 cells were added to each fiber piece (corresponding to a quarter of what is obtained in each tube), and incubated in a 96 well for 1 h before transfer to a 24 well with 1 ml fresh media.

### Foam Formation

The silk foam scaffolds were made with 20-40 μl of silk protein (3 mg/mL) that was placed in the middle of a hydrophobic culture well. Air was pipetted into the 20 ul protein drop for 30 times. Cell suspensions (0.5-2 million cells/ml) were prepared in respective culture media containing 25 mM Hepes but without serum and added dropwise (10-20 μl) either before or after introduction of air bubbles. The cell containing foam plates were incubated for 30-60 minutes in the cell incubator before the appropriate cell culture medium was added.

Film Formation

Silk protein (3 mg/mL) was centrifuged after thawing to remove aggregates. 5 or 10 μL of protein solution was added to a hydrophobic culture well (Sarstedt suspension cells), to create a drop of liquid on the surface of the well bottom. Thereafter, an equal volume of cell suspension (HDFn or HaCaT, 0.5 milj/mL, 1 milj/mL or 2 milj/mL) was added to the drop of protein. The cell-containing films were incubated 30-60 min in the cell incubator followed by 30 min (5+5 μL films) or 60 min (10+10 μL films) in the LAF bench without lid, before 1 mL of culture medium was added. Culture was conducted for 2 or 3 days before Live/Dead assay (Life Technologies) was performed.

3D Foam Formation with Hepatocytes and Glioma-Forming Cells

Recombinant spider silk protein is used to prepare a foam of 20 μl of the protein (3 mg/mL), placed in the middle of a well in a 24 well plate. Air is pipetted into the 20 μl protein drop. A cell suspension (1 million cells/ml) is prepared in DMEM containing 25 mM Hepes without serum (Invitrogen). A final amount of 20 000 cells (20 μl) from the prepared cell suspension is carefully put on top of the foam with small drops. The cell-containing foams are incubated for 1 h in the cell incubator before more RPMI-1640 medium supplemented with 10% FBS is added (500 μl, Invitrogen).

Analysis of Cells within Silk Scaffolds

### Proliferation

Alamar Blue (Invitrogen, Stockholm, Sweden) was used to investigate viability and proliferation of incorporated cells in the fibers and foam over a period of up to 21 days. Alamar Blue was diluted 1/10 in the appropriate cell culture medium and added to each well containing fibers or foam and incubated for 2 hours in the cell incubator. After incubation, the supernatants were transferred to new 96-well plate (Corning) and OD was measured at 595 nm using a multimode plate reader (ClarioStar, LabVision). OD was plotted as fluorescent intensity per well. The culture was then, after Alamar Blue incubation and removal, continued with fresh complete medium.

BrdU (Invitrogen) was added to a final concentration of 10 μM at day 3, 10 and 14 of culture of the cell-containing silk scaffolds and incubated for 20 h with BrdU before wash, fixation and cryosectioning. DNA denaturation was performed in 1 N HCl in ice for 10 min, 2 N HCl at RT for 10 min followed by 20 min at 37° C. Neutralization was done immediately in 0.1 M Borate buffer pH 8.5 for 10 min at RT. Samples were washed 3×5 min in PBS (pH 7.4) with 0.1% Triton X-100, and blocked 15 min in PBS/1% BSA. Staining was done with BrdU-Mouse Monoclonal Antibody (Clone MoBU-1), Alexa-488 conjugated (Molecular Probes B35130) at 4 pg/mL in PBS/1%BSA for 1 h at RT (or overnight at +4° C.). Counterstain was done with DAPI. Slides were mounted in Fluorescence mounting medium (Dako). Micrographs were taken at 10× and 20× in Nikon inverted fluorescence microscope.

### Viability

Live/Dead cell viability assay (Molecular Probes/Invitrogen, Stockholm, Sweden) was performed on the cell-containing silk scaffolds at selected endpoint, after 7-21 days of culture. The silk scaffolds were washed in PBS before a mixture of Calcein (1/2000) and EthD-1 (1/500) in PBS was added to the wells and incubated for 30 minutes in RT. Staining was then analyzed for live (green) and dead (red) cells in a fluorescent inverted microscope (Eclipse, Nikon, Sweden). Images were taken at 10× magnification at selected planes of the scaffolds. Using the software NIS-elements 3 equal areas per image was calculated for % viability (amount of green cells/total amount of cells ×100).

### Cell Spreading and Attachment

After gentle washing, cell-containing silk scaffolds were fixed with 4% paraformaldehyde, permeabilized with 0.1% Triton X-100 in PBS, and blocked with 1% bovine serum albumin (BSA, AppliChem) in PBS. Primary antibody mouse anti human vinculin (Sigma V9131) was used at a concentration of 9.5 μg/ml in 1% BSA. Secondary antibody was AlexaFlour488 goat anti mouse IgG (H+L), cross adsorbed (Invitrogen), used at 1:500. Phalloidin-AlexaFluor594 (Life Technologies) was used at 1:40 to detect filamentous actin. DAPI was used for nuclear staining. Slides were mounted in fluorescence mounting medium (Dako, Copenhagen). The stained cells were analyzed using an inverted microscope (Nikon Eclipse Ti) at 4× and 10× magnification.

### Cell Distribution and Morphology

At the endpoint, the cell-containing silk scaffolds were fixed in 4% paraformaldehyde for 15-30 minutes washed, incubated in 20% sucrose until embedded in Tissue-Tek (Sakura, Japan), cryo-preserved and sectioned in a cryostat to 12-25 μm thick consecutive sections. Selected sections were morphological evaluated after following a standard Heamatoxylin and Eosin (HE) staining for frozen tissue.

### Differentiation

Selected sections of cell-containing silk scaffolds were permeabilized in 0.5% Triton ×100 for 5 minutes, blocked with 5% normal goat serum in PBS for 30 minutes at RT and stained for Desmin (Anti-Des, Prestige Antibodies, Atlas Antibodies, Sigma Aldrich, 1:200). Next the fiber sections were probed with a secondary antibody raised in goat against rabbit coupled to Alexa488 (Molecular Probes, 1:1000).

### Collagen Production

Selected sections were blocked with 1% BSA in PBS before staining with mouse anti collagen type I (clone COL-1, SigmaAldrich) at 3.5 mg/mL in 1% BSA, followed by AlexaFluor488 goat anti mouse IgG antibody (Invitrogen). DAPI was used for nuclear staining. Slides were mounted in Fluorescence mounting medium (Dako). Micrographs were taken at 10× in Nikon inverted fluorescence microscope.

### Insulin Production and Secretion

Silk foam scaffolds with clusters of endocrine cells were washed in PBS and fixed in 1% paraformaldehyde and thereafter permeabilized in PBS containing 0.3% Triton ×100 for 15 min. Blocking was done with 6% fetal calf serum (FCS) in PBS containing 0.1% Tween for 1 h at room temperature (RT). The samples were then incubated with antibodies against insulin (guinea-pig anti-insulin, 1:1000, Dako), rabbit anti-human CD44 (1:100) and/or mouse anti-human CD31 (1:100, BD Pharmingen) overnight at 4° C. The next day the samples were probed with a secondary antibody raised in goat against guinea-pig coupled to Alexa488 and rabbit and mouse coupled to Alexa 594 (Molecular Probes, 1:1000).

Endocrine cell clusters from MIP-GFP transgenic mice were cultured together with hMSC and HDMEC for 7 days in 24-well plates in foam consisting of a mixture of 2R and FN protein. The foam was gently put on top of a 0.5 ml column packed with Bio-Gel P4 polyacrylamide beads (Bio-Rad). Dynamics of insulin release were studied by perifusing the clusters at 37° C. with the Hepes buffer with 3 mM glucose as a basal condition and 11 mM as a stimulatory glucose concentration for insulin release, followed by 25 mM KCl. The flow rate was 40 ml/min, and 2 min fractions were collected and analyzed for insulin with an insulin assay HTRF kit (Cisbio).

### Mechanical Analysis of Cellular Silk Scaffolds

Stress versus strain (% length extension) of cell-containing fibers was measured under physiological-like conditions (37C, 1×PBS) with a custom built Zwick/Roell Material testing machine using a ramp force of 0.2 N/min. The fiber ends were mounted with specimen grippers. Fibers with macroscopic defects or that were obviously maltreated during mechanical testing were excluded. A circular initial cross section of the fibers was used for the calculation of stress.

### Transplantation and in Vivo Imaging of Cellular Silk Constructs

Transplantations were done using essentially the same procedure as previously described in Speier S, et al. Nat Protoc. 3:1278-1286 (2008). Cell-containing silk matrices were dissected into smaller pieces (˜50 um) and put into sterile culture media before aspiration into a 27 gauge eye cannula (prepared by adapting a blunt ended patch clamp glass capillary) connected to a 1 mL Hamilton syringe (Hamilton) via 0.4-mm polyethylene tubing (Portex). B6 Albino A++ (C57BL/6NTac-Atm1.1Arte Tyrtm1Arte, Taconic, Cologne, Germany) purchased from Jackson Laboratory (Bar Harbor, Me., USA) were used as recipients after anesthesia with 2% isoflurane (vol/vol). When the cannula had been stably inserted into the anterior chamber, the transplants were slowly injected in the smallest volume possible of sterile saline solution into the anterior chamber, where they settled on the iris. Analgesia was obtained after surgical procedures with buprenorphine (0.05-0.1 mg/kg s.c.).

In vivo imaging of scaffolds in the anterior chamber of the eye of the transplanted animals was performed essentially as previously reported in Speier S, et al. Nat Protoc. 3:1278-1286 (2008). Briefly, mice were anesthetized with 2% isoflurane air mixture and placed on a heating pad, and the head was restrained with a head holder. The eyelid was carefully pulled back and the eye gently supported, Viscotears (Novartis) was used as an immersion liquid between the eye and the objective. Scanning speed and laser intensities were adjusted to avoid cellular damage to the mouse eye.

### Results

Formulation of Silk Scaffolds with Integrated Cells

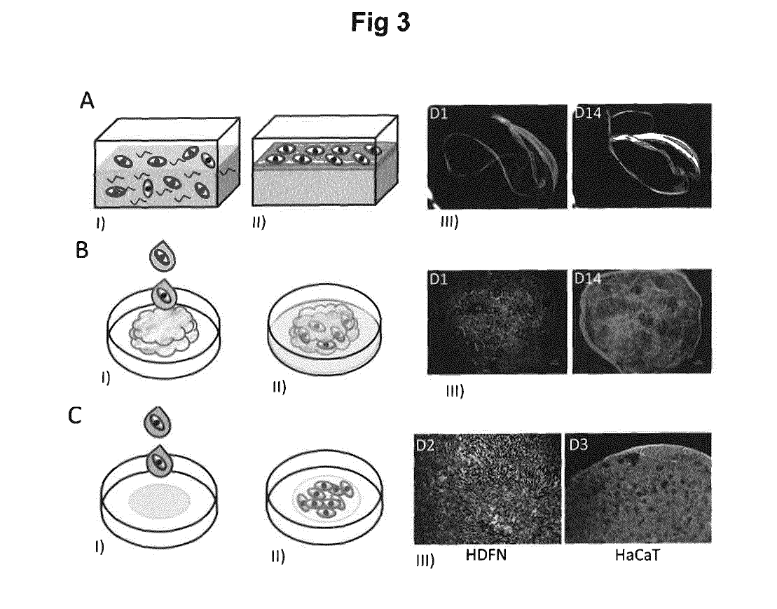

FIG. 3 shows a schematic description of formulation of silk scaffolds with integrated cells.

### Fiber Formation

FIG. 3A shows a schematic description of formulation of cell-containing silk fibers. The silk protein is mixed with cells suspended in media (I). During gentle wagging for 1-3 hours incubation the silk protein assemble at the air-liquid interface into a fibrous mat with incorporated cells (II). The cell-containing silk fibers are then easily retrieved and placed in a culture well (III).

Gentle wagging of silk protein solution mixed with cells in culture media in a tube resulted in formation of visible fibers within 20 minutes, thus within the same time frame as for silk protein alone. The fiber formation was allowed to continue for 1-3 hours before transferring the fiber bundles to cell culture wells with fresh media. To the naked eye the cell-containing fibers look very similar to ordinary silk fiber bundles at day 1 (FIG. 3A), but continue to grow in thickness during the culture period. For some cell types, such as fibroblasts and skeletal muscle satellite cells, the smaller fiber bundles were typically curled up after a few days in culture. This could be avoided by mounting them as elongated fiber bundles between two fixed points using an insert in the well.

Due to sedimentation, a substantial fraction of the cells was found at the bottom of the tube during fiber formation. In order to avoid this cell loss, an inverted set up was developed, with an oil phase of higher density underneath the silk/cell solution. Using this approach the cell containing fibers were formed at the buffer:oil interface were the cells were trapped, instead of at the air:buffer interface. A higher cell density within the fibers was obtained using this method, although at the cost of more irregular morphology.

### Foam Formation

FIG. 3B shows a schematic description of formulation of cell-containing silk foam. The silk protein solution with cells in media is transformed into wet foam (I) by gently introduction of air. After 30-60 minutes pre-incubation, additional culture media is added to cover the foam (II). The cellular silk foam can then be cultured in the well (III). Scalebar=1 mm.

Gently introduction of air bubbles into a mixture of silk protein solution and cells in culture media gave rise to an expanding foam structure, to the naked eye similar to what is accomplished with only silk protein (FIG. 3B). When fresh cell culture media is added after a pre-incubation period of 30-60 minutes the foam holds together as a coherent three-dimensional structure. Throughout the culture period the foam gets increasingly white and less transparent.

### Film Formation

FIG. 3C is a schematic description of formulation of cell-containing silk film. The silk protein solution is placed as a drop into a culture well, where after cells suspended in media are directly added drop wise (I). After 30-60 minutes of pre-incubation, additional culture media is added to cover the film (II). The cell-containing silk film can then be cultured in the well and subjected to L/D staining (III). Left: 4× magnification of HDFn (20 000cells/film) after 2 days, and right: 4× magnification of HaCaT (10 000 cells/film) after 3 days.

By addition of cells in culture media into a defined drop of silk protein solution the cells stay together as a coherent film if pre-incubated for 30-60 minutes before fresh culture media is added. Depending on the amount of cells added, the cell-containing film gets confluent within 1-3 days of culture.

Cells Maintain Proliferative Capacity within Silk Scaffolds

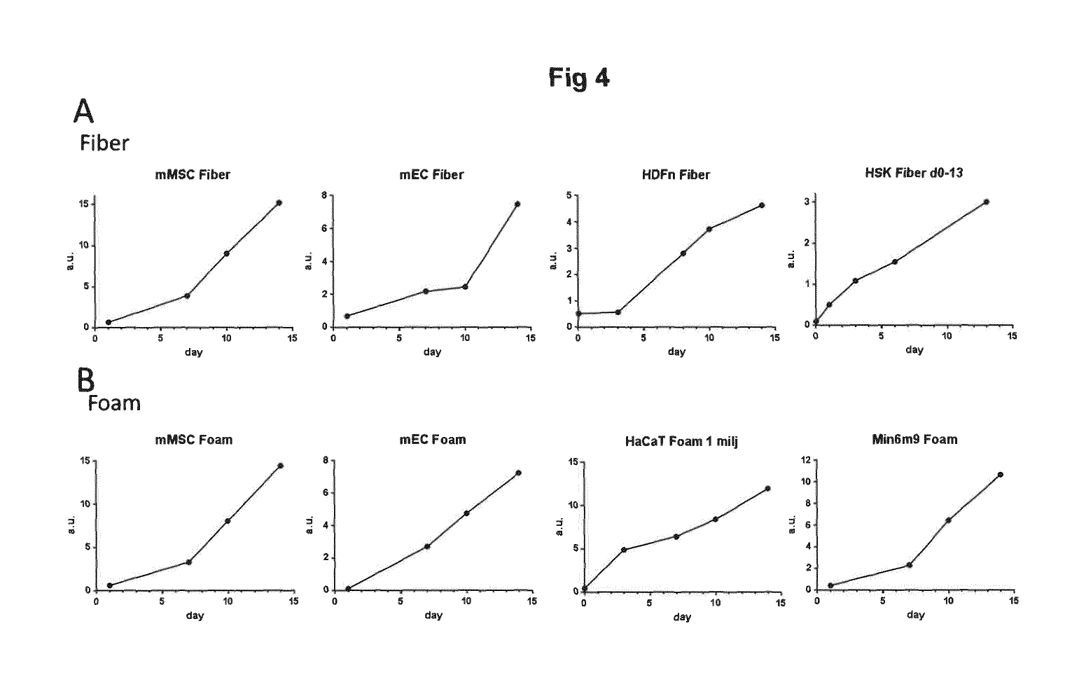

Measurements of cell proliferation (using Alamar blue cell viability assay) confirmed a growth profile of proliferating cells within both foam, fibers and films. FIG. 4 shows metabolic activity of cells within silk scaffolds. FIG. 4A shows representative growth profiles of individual silk fiber bundles containing different cell types (mMSC, mEC, HDFn, Hsk) measured using the Alamar blue viability assay. FIG. 4B shows representative growth profiles of individual silk foams containing different cell types (mMSC, mEC, HaCaT, MIN6m9) measured using the Alamar blue viability assay.

The amplitude of the signal varied between samples of fiber bundles, probably reflecting an uneven distribution of captured cells. This could partly be avoided using a higher cell density and quick handling before initiated fiber formation. For the foam and film format the growth profiles were more reproducible between samples, probably due to the fact that here all added cells are directly captured within the scaffolds.

The slope of the growth curves was affected by both the cell density and cell type used. Typically, a slower initial phase could be observed, followed by a steeper curve. Samples which reached a high plateau after two weeks typically contained confluent cell layers, as could be confirmed with cellular stainings (see below).

To examine if cells incorporated within the silk scaffolds (and not just cells on the surface) are dividing and proliferating, we also performed BrdU analysis. By adding BrdU to the medium the last 20 h before fixation, cells that undergo cell division will incorporate BrdU molecules in their genome during DNA-synthesis. These BrdU molecules can then be detected by immunofluorescence. In this way, proliferating cells present deep within the silk fibers could be demonstrated at all time points examined (d4, d11 and d15). The ratio of dividing cells was higher at the earlier time points, and decrease during culture period (d4 80%, d11 50%, d15 25%) which is normal for in vitro culture where the cells get confluent.

The Majority of the Cells are Viable within the Silk Scaffolds

The viability of the cells within the silk scaffolds were analyzed with microscopy using a two-color fluorescence viability assay which simultaneously stains live (green) and dead (red) cells.

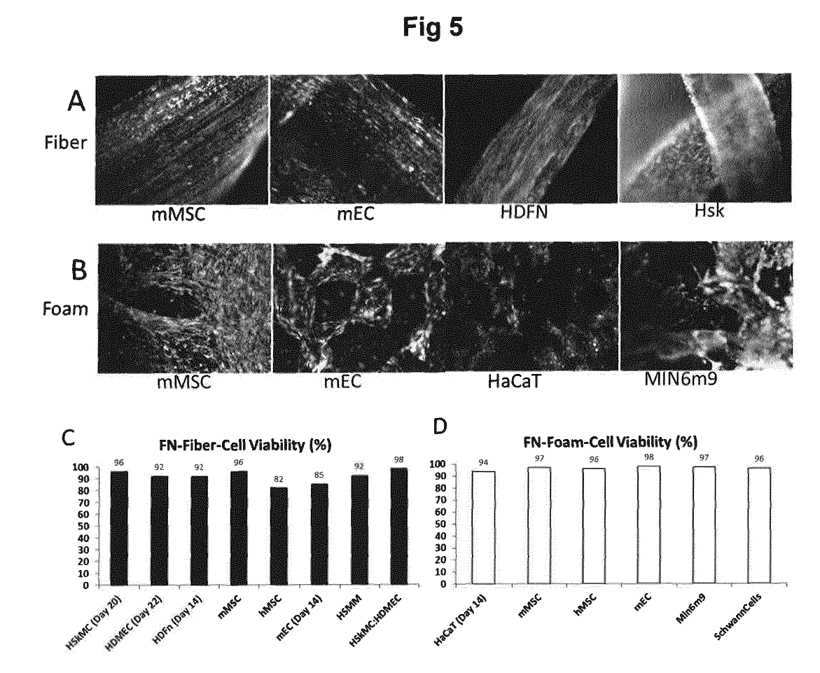

FIG. 5 shows viability of cells within silk scaffolds:
A) Live staining of various cell types within cellular silk fibers (10×).
B) Live staining of various cell types within cellular silk foam (10×).
C) Viability of cells within fibers.
D) Viability of cells within foam.

Although the scaffolds to the naked eye looked like ordinary silk materials, although a bit thicker, it became evident under the fluorescence microscope that the samples contained a substantial amount of cells, with the major fraction alive (FIG. 5). The viability within all fibers was above 80% (FIG. 5C) and well above 90% for all foam scaffolds (FIG. 5D). For film, the viability was very depending on the amount of cells added, with a viability of 80-90% if cells were added above the number of cells possible to fit in a confluent layer (data not shown).

The Cells Spread Out and Attach via Focal Adhesions within Silk Scaffolds

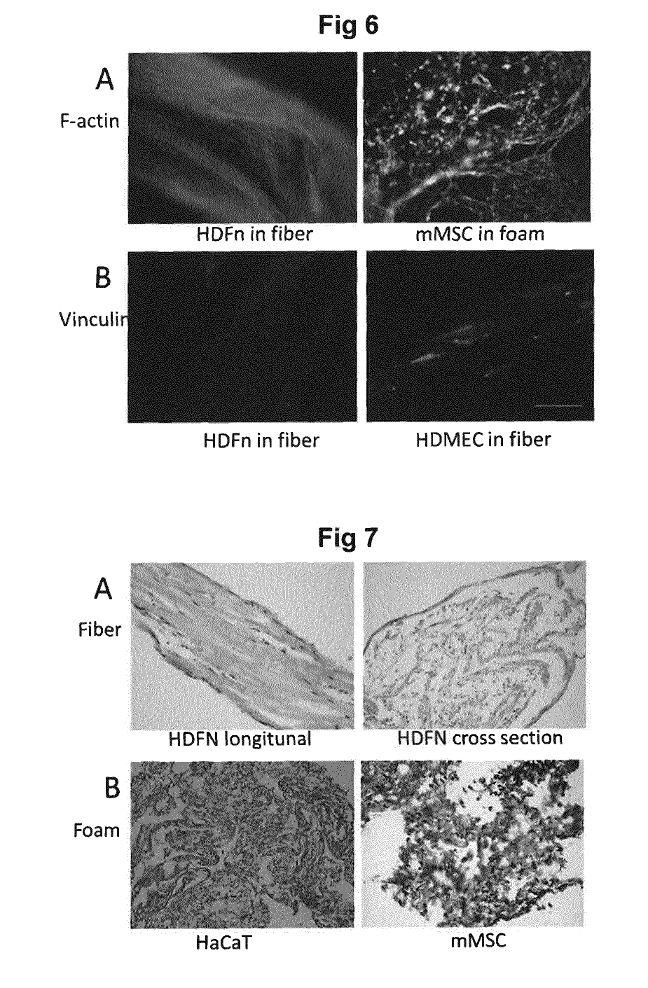

The ability of the cells to stretch and spread out within the silk scaffolds was evaluated by staining for stress fibers (via actin filament). FIG. 6 shows spreading of cells within silk scaffolds. FIG. 6A shows f-actin staining of HDFn cells within fibers (left) and Dapi (round spots represents nuclei) and f-actin staining of mMSC in foam (right) (10×). FIG. 6B shows f-actin and Vinculin (bright spots) staining of HDFn (left) and HDMEC (right) in fibers.

In the fiber format the cells were found along the fiber bundle, with mostly elongated cell shapes (FIG. 6A, left). In the foam format the cells are typically found stretched out and rambling between the silk structures (FIG. 6A, right). Well-organized actin stress fibers can be seen in the majority of the cells within both fibers and foam.

Cell attachment via formation of focal adhesions was analyzed after staining for F-actin in combination with vinculin, which is one of the major components of the focal adhesion complex, often situated close to the cell membrane. Co-staining of F-actin and vinculin is thus a sign of integrin-involved, well established binding of cells to the scaffold. Within the cell containing fibers it was possible to distinguish focal adhesion points as bright spot at the edges of elongated cells (FIG. 6B). Within the foam scaffolds the cells were distributed randomly in three dimensions, which complicated distinction of focal adhesion points, although clear signal from vinculin staining could be found (data not shown).

Cells are Distributed throughout the Silk Scaffolds

In order to confirm that the cells are well distributed within the silk scaffolds we performed cryosectioning and H/E staining to locate cells. The fibers were sectioned both longitudinal and cross the fiber axis (FIG. 7A). Cells could be seen throughout the fiber, although some areas were more populated than others. In accordance with results from the viability assays, the foam scaffolds were more densely populated with cells throughout the material (FIG. 7B).

FIG. 7 shows distribution of cells within silk scaffolds. FIG. 7A shows H/E staining of longitudinal (left) and cross (right) cryosections of silk fibers with HDFn cells. Dark spots represent nuclei. FIG. 7B shows H/E staings of cryosections of cellular silk foams with HaCaT (left) and mMSC (right). Dark spots represent nuclei.

Silk Scaffolds with Cells are Mechanically Stable

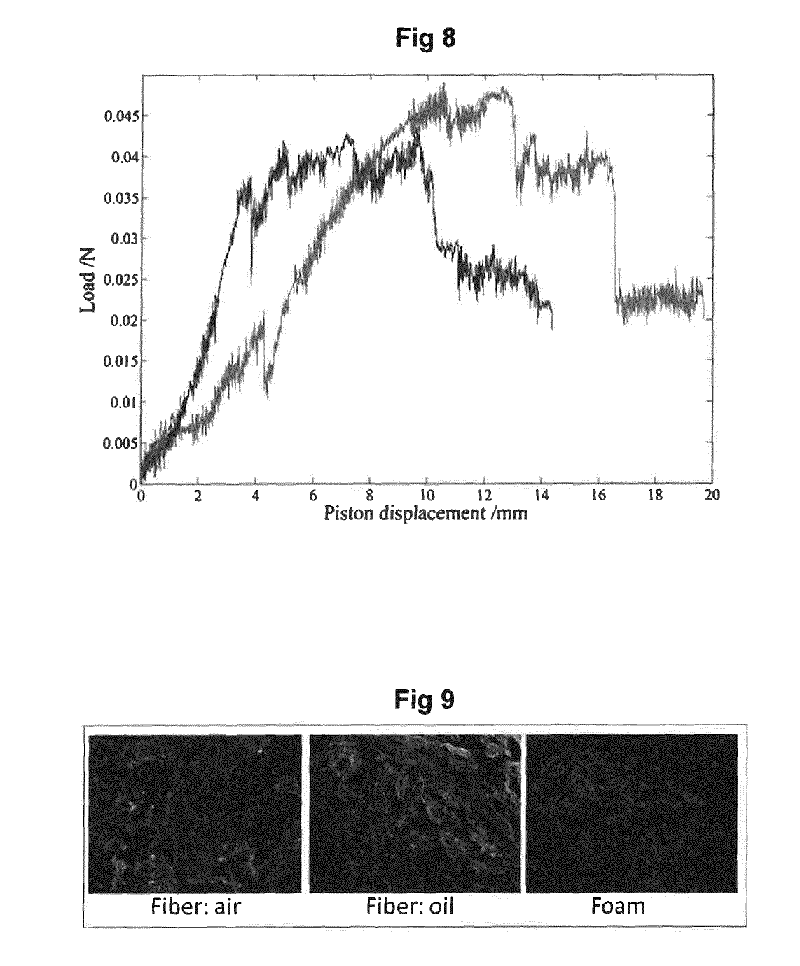

The cell-containing silk scaffolds were stable enough for handling throughout the culture period and analysis procedures, resembling of ordinary silk scaffolds in terms of flexibility under humid conditions. In order to relate the mechanical properties in comparison to native tissue, the cell containing fibers were subjected to tensile testing in pre-warmed physiological buffer (FIG. 8). After an initial elastic phase, the deformation zone was reached and the fibers were extended to approximately twice its initial length.

FIG. 8 shows mechanical properties of silk fibers with cells by stress strain curves of two representative silk fibers with fibroblasts (HDFn) cultured for two weeks.

Fibroblasts Produce Collagen within Silk Scaffolds

As a first step to confirm that the cells maintain their main functions during culture within the silk scaffolds, it was investigated if fibroblasts produced collagen type I when growing within the different scaffold types. By staining intracellular collagen type I, it was evident that a majority of the cells produced collagen although within fiber or foam.

FIG. 9 shows immunofluorescence staining of collagen type I. Silk scaffolds with fibroblasts cultured for two weeks before stained with collagen type I specific antibodies for the detection of native helical collagen type I. The specific antibody detects both intracellular and extracellular collagen. Round spots represents Dapi staining of nuclei.

Cells within Silk Scaffolds can be Differentiated

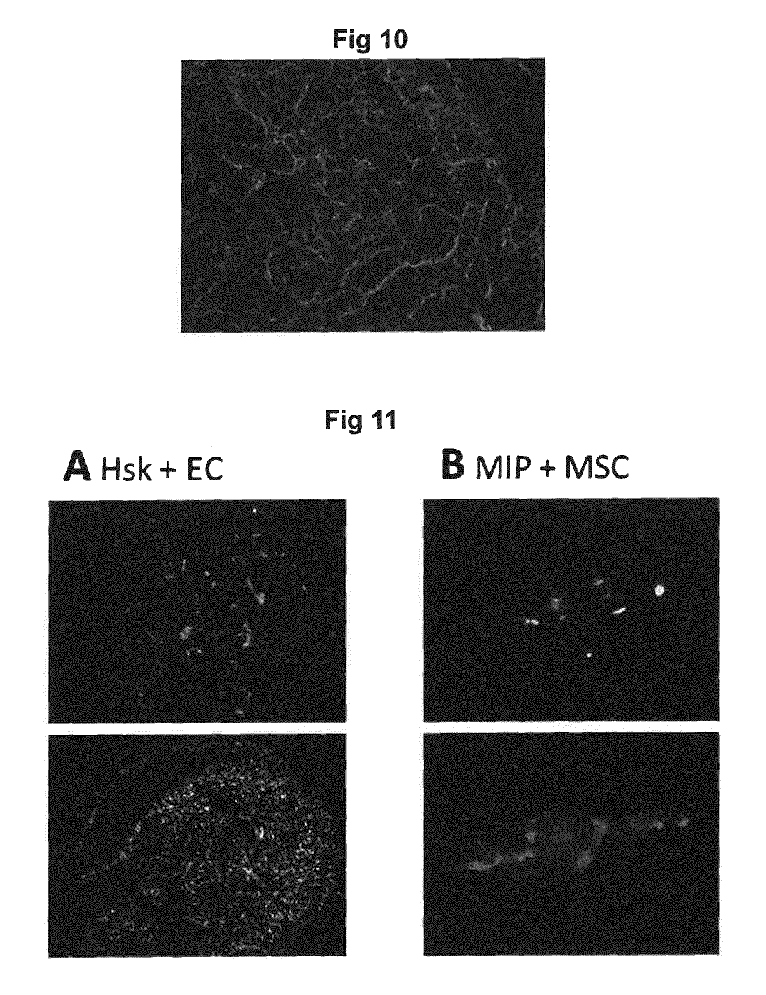

In order to confirm that cells within the silk scaffolds are accessible for differentiation, fibers with human skeletal muscle satellite cells were transferred to DMEM culture media to promote differentiation. Staining of Desmin was applied to visualize myotube formation (FIG. 10).

FIG. 10 shows immunofluorescence staining of myotube formation. Fibers with Hsk cells cultured for two weeks and thereafter kept in diffentiation media for another two weeks, before staining with Desmin. Round spots represent Dapi staining of nuclei.

Several Cell Types can be Co-Cultures within Silk Scaffolds

Most native tissue types consist of several cell types organized together in a complex three-dimensional arrangement with extracellular matrix surrounding the cells and keeping them together. In order to replicate this in engineered tissue constructs it is therefore of importance to achieve co-cultures within the scaffolds. With the herein described method for formulation of cell containing silk scaffolds it is practically very easy to combine several cell types, as long as they can be cultured in a similar media.

We have herein demonstrated an example of co-culture in silk fiber using human skeletal muscle satellite cells and endothelial cells (FIG. 11A). The endothelial cells were found distributed with some local clusters within the fibers, possibly representing an early state of vessel formation.

As example of co-culture within silk foam, we have combined endocrine cells with supportive mesenchymal stem cells and endothelial cells (FIG. 11B).

FIG. 11 shows presence of several cell types co-cultured within silk scaffolds. FIG. 11A shows a section of a silk fiber subjected to co-culture and immunostained for EC (upper) and Hsk cells (lower). FIG. 11B shows silk foam subjected to co-culture and immunostained for MIP (upper) and MSC (lower).

Endocrine Cells within Silk Scaffolds Maintain Functional

The endocrine cell islets found within the pancreas, often called islets of Langerhans, is a typical example of cells which require the right cellular neighbors as well as a physical three-dimensional support in order to stay functional.

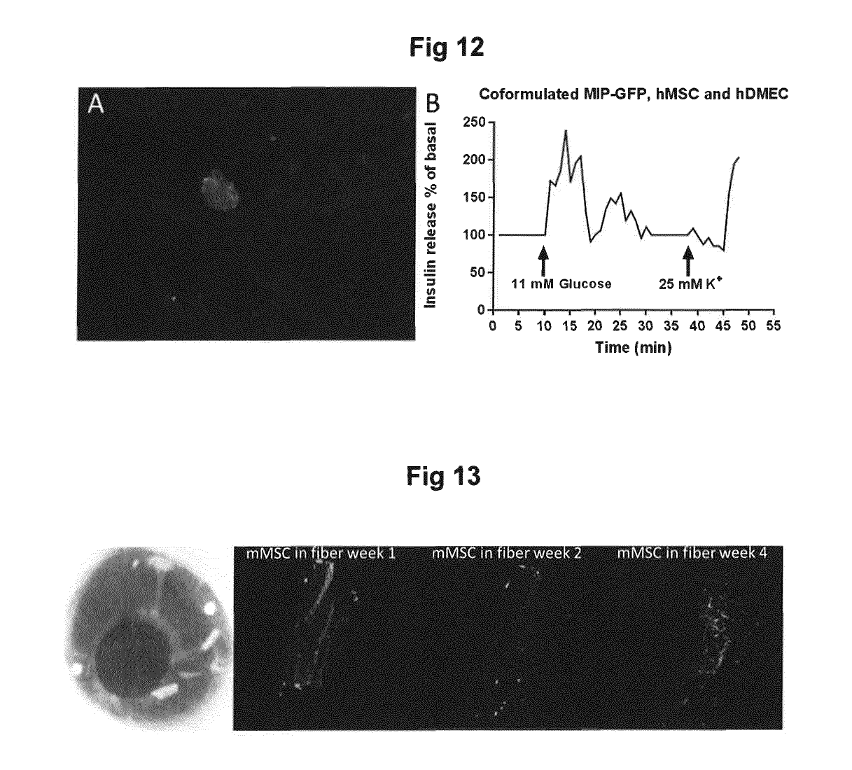

FIG. 12 shows that islet-like clusters are functional within silk scaffolds. FIG. 12A shows insulin staining of endocrine cells and a cluster thereof within a silk foam. A solution of dispersed endocrine cells, retrieved by cell dissociation of isolated islets, has a tendency to cluster into islet-like shapes if cultured within the silk foam. Staining for insulin confirm that the single cells as well as clusters maintain their ability to produce insulin within the silk foam (FIG. 12A).

To further elucidate if the islet-like clusters formed within the silk foam were functional, i.e. produced insulin only upon stimulation, the amount of insulin was measured after stimulation with physiological concentrations of glucose. FIG. 12B shows a representative curve of dynamic insulin release after perifusion of islet-like clusters within silk foam. The insulin values are normalized for dsDNA, and the insulin values in the chart are presented as % of basal level. In order to imitate a physiological stimulation as far as possible, the clusters were dynamically stimulated with increasing glucose levels. Silk foam containing islet-like clusters were put into a column that was dynamically perifused by pumping through buffers with different concentrations of glucose. Thereby, an increase in insulin release after stimulation with high concentration (11 mM) of glucose could be measured, which was reversed when the glucose concentration was brought back to basal levels (3 mM) (FIG. 12B). Moreover, the clusters within the silk foam also responded to subsequent KCl stimulation.

In Vivo Imaging of Silk Scaffolds with Cells

Next, it was investigated how cell-containing silk scaffolds would persist in vivo. Cells were first cultured within fibers and foam respectively, and were after 1 week transplanted into the anterior chamber of the eye of a mouse. The open window offered by the eye was utilized for evaluation of the silk scaffold using a camera (FIG. 13, left) and the cells (in vivo traced) therein using confocal microscopy (FIG. 13). The macroscopic appearance of the silk scaffolds was similar for all four weeks in vivo, while the distribution and amount of cells slowly changed, probably due to cell migration as well as degradation.

FIG. 13 shows in vivo imaging of silk scaffolds with cells. To the left is shown a picture of an eye with cell-containing (mMSC) fibers (in white) transplanted into the anterior eye chamber. To the right are representative confocal micrographs of in vivo traced cells (mMSC) within a silk fiber after 1, 2 and 4 weeks in vivo.

Integration of Cells Depend on when they are Added to the Silk Protein

Alternative formulation protocols were investigated to determine how the cells are distributed within the silk scaffolds depending on at which stage they are added during the formulation process.

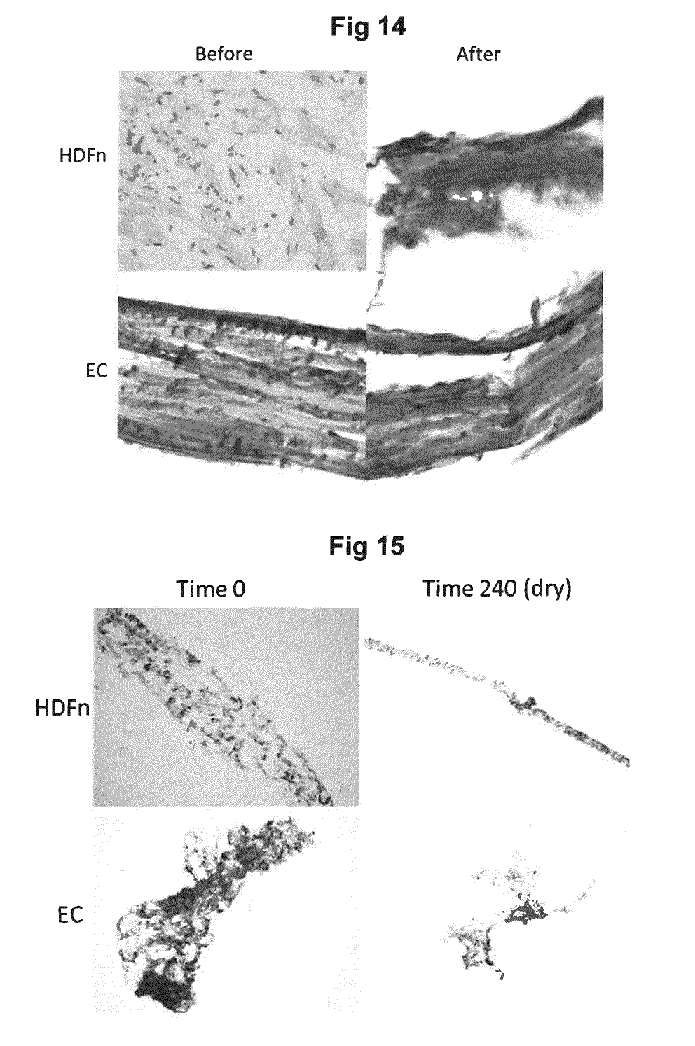

Fiber formation occurs at a hydrophilic/hydrophobic interface within a tube put on incubation during gentle rocking. In order to maintain sterile conditions, the tube has to be closed during incubation, why there are only two options for cell addition: either to the silk protein solution before fiber formation has initiated, or on top of the formed fibers after they have been retrained and put in a culture well. Since the fibers form as a bundle, there are some cell penetrations possible also when cells are added after fiber formation (FIG. 14, right column). However, if the cells are added to the silk protein solution before fiber formation, a more even distribution of cells within the fibers is obtained (FIG. 14, left column).

FIG. 14 shows cell distribution within silk fibers. H/E staining of cryosections of silk fibers with HDFn (upper row) and EC (lower row) added before (left column) or after (right column) fiber formation. Dark spots represent cell nuclei.

Foam formation is achieved by gently introduction of air bubbles into a silk protein solution. The silk scaffold slowly solidifies at the interface in each air bubble. If the cells (in media) are added directly to the silk protein solution before introducing air bubbles, they get evenly distributed throughout the silk foam. If the cells are added dropwise after formation of the foam, the cells in media slowly spread through the foam structure as long as the foam is still wet; with more evenly distribution the earlier the cells are added. If the cells are added to dry foam, the foam structure partly collapses, resulting in a thinner and more net-like structure of the silk.

Foam scaffolds with cells added at different time points were stained for f-actin (to visualize cells) and imaged using an inverted fluorescence microscope. Distinct and different cells could be seen in several z-plans of all analyzed foam scaffolds were the cells had been added before drying (0-90 min) (Table 3). For foam scaffolds that were allowed to dry before adding the cells it was only possible to distinguish one z-plan with cells.

TABLE 3
Analysis of silk foam scaffolds with addition of cells at different time points

| Time point for | Number of z-plan | Number of cells | Time point | Number of z-plan | Number of cells |
|---|---|---|---|---|---|
| addition of | with different | in layer (H/E | for addition | with different | in layer (H/E |
| endothelial | cells (fluorescence | staining of | of fibro- | cells (fluorescence | staining of |
| cells (min) | microscope) | cryosection) | blasts (min) | microscope) | cryosection) |
| 0 | 3 | 10-15 | 0 | 3 | 6-8 |
| 10 | 3 | 10 | 10 | 3 | n.a. |
| 60 | 3 | 5 | 60 | 2 | 3-5 |
| 90 | 4 | n.a. | 90 | 3 | 3-5 |
| 240 (dry) | 1 | n.a. | 240 (dry) | 1 | 1-2 |

The foam scaffolds were further investigated by cryosectioning (from the side) and stained with H/E. For all analyzed foam scaffolds were the cells had been added before drying (0-90 min) the scaffold had a poofy appearance, with several cells in layers (FIG. 15, left column). Foam scaffolds that were allowed to dry before addition of cells, most cells were located as a thin and compact line, with one or maximum two cells layers (FIG. 15, right column).

FIG. 15 shows cell distribution within silk foam. H/E staining of cryosections of silk foam with HDFn (upper row) and EC (lower row) added to the silk protein solution at time 0 (left column) or after drying for 240 minutes (right column).

### Example 2

### Integration of Cells into Foam of Minispidroin with an Alternative C-Terminal Domain

Production and purification of the silk protein FN_{cc}-RepCT_{MiSp} (SEQ NO: 69) was done as described in Example 1. CT_{MiSp} (SEQ ID NO: 68) is a minor ampullate spider silk protein derived from Aranaeus ventricosus.

Primary endothelial cells from capillaries of human origin (HUVEC, PromoCell) were cultured in Endothelial cell growth medium MV2 (PromoCell) containing fetal bovine serum (FBS, 5%). The cells were used at passage 6.

Silk foam scaffolds were made with 20-40 μl of the silk protein (3 mg/mL) that was placed in the middle of a hydrophobic culture well. Air was pipetted into the 20 μl protein drop 30 times. Cell suspensions (0.5-2 million cells/ml) were prepared in respective culture media containing 25 mM Hepes but without serum and added dropwise (10-20 μl) directly after introduction of air bubbles. The cell containing foam plates were incubated for 30-60 minutes in the cell incubator before the appropriate cell culture medium was added.

Alamar Blue (Invitrogen, Stockholm, Sweden) was used to investigate viability and proliferation of incorporated cells. Alamar Blue was diluted 1/10 in the appropriate cell culture medium and added to each well containing foam and incubated for 2 hours in the cell incubator. After incubation, the supernatants were transferred to new 96-well plate (Corning) and OD was measured at 595 nm using a multimode plate reader (ClarioStar, LabVision). OD was plotted as fluorescent intensity per well. The culture was then, after Alamar Blue incubation and removal, continued with fresh complete medium.

Live/Dead cell viability assay (Molecular Probes/Invitrogen, Stockholm, Sweden) was performed on the cell-containing silk foam after 8 days of culture. The silk scaffolds were washed in PBS before a mixture of Calcein (1/2000) and EthD-1 (1/500) in PBS was added to the wells and incubated for 30 minutes in RT. Staining was then analyzed for live (green) and/or dead (red) cells in a fluorescent inverted microscope (Eclipse, Nikon, Sweden). Images were taken at 4× magnification at selected planes of the scaffolds.

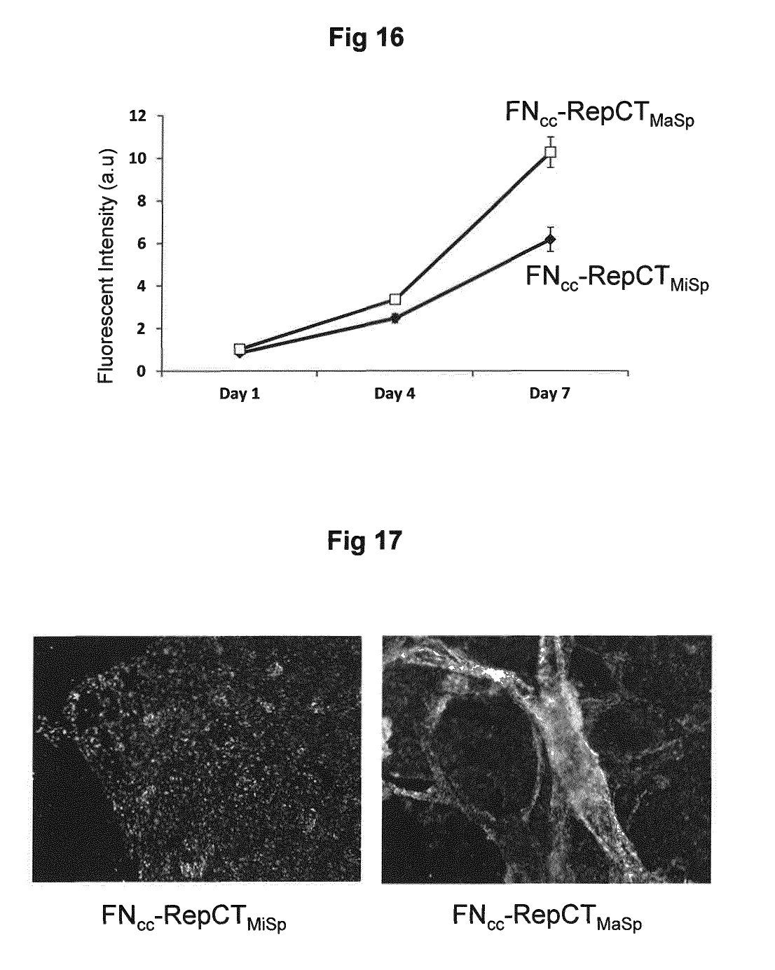

FIG. 16 shows a growth curve (n=3, SEM) of proliferating cells (20 000 HUVEC/well) within foam of FN_{cc}-RepCT_{MiSp} (SEQ ID NO: 69; filled diamonds) and the corresponding FN_{cc}-RepCT_{MaSp} (SEQ ID NO: 27; open squares), confirming similar proliferation.

FIG. 17 shows live cell staining at the end of the culture (Day 8) and confirms the presence of viable cells integrated within the foams (4× magnification) of both FN_{cc}-RepCT_{MiSp} (left panel) and FN_{cc}-RepCT_{MaSp} (right panel).

### Example 3

### Integration of Cells within Matrices of Silk Fibroin from the Silk Worm Bombyx mori

Pieces of silk cocoons from B. mori were degummed in boiling 0.02 M sodium carbonate, washed properly with distilled water, and dried overnight at room temperature. Degummed and dried silk were then dissolved in 9.3 M LiBr and dialyzed against milli-Q water using dialysis membrane (MWCO 12 kDa) for 3 days with successive water change.

For fiber formation, fibroin protein (0.5-10 mg) was mixed with 0.5-2 million cells in respective culture media in a total volume of 4 ml. The fiber formation together with cells was performed in RT under gentle wagging for 1-24 hours. The formed fibers were then washed in 1×PBS and thereafter transferred into non-treated 24-well plates and further kept in culture by adding fresh media.

For foam formation, 20-40 μl of fibroin protein (3 mg/mL) was placed in the middle of a hydrophobic culture well. Air was pipetted into the 20 μl protein drop 30 times. Cell suspensions (0.5-2 million cells/ml) were prepared in respective culture media containing 25 mM Hepes but without serum and added dropwise (10-20 μl) either before or after introduction of air bubbles. The plates were incubated for 30-60 minutes in the cell incubator before the appropriate cell culture medium was added.

For film formation, 5 or 10 μL of fibroin protein solution (3 mg/mL) was added to a hydrophobic culture well (Sarstedt suspension cells), to create a drop of liquid on the surface of the well bottom. Thereafter, an equal volume of cell suspension was added to the drop of protein. The cell-containing films were incubated 30-60 min in the cell incubator followed by 30 min in the LAF bench without lid, before 1 mL of culture medium was added.

Cells were treated and cultured as described under Example 1. The Alamar blue and Live/dead viability assays were performed as described under Example 2.

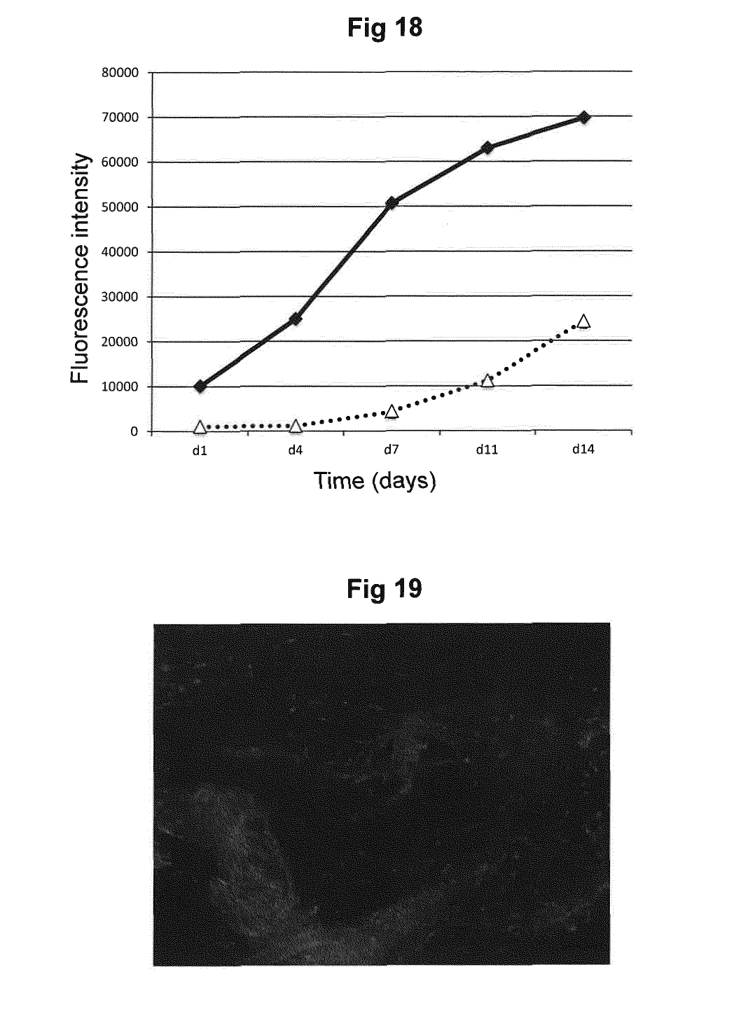

FIG. 18 shows the growth curve of proliferating cells (hDF) within a fiber of B. mori silk fibroin (open triangles, dotted line), compared to corresponding fiber of FN_{cc}-RepCT (SEQ ID NO: 27; filled diamonds, solid line). FIG. 19 shows live staining of fibroblasts (HDFn, ECACC, P7; scale bar 250 μm) integrated within fibers of B. mori silk fibroin and further confirms presence of viable cells at day 15.

The presence of viable HUVECs within a foam of B. mori silk fibroin was determined after 19 days of culture (data not shown).

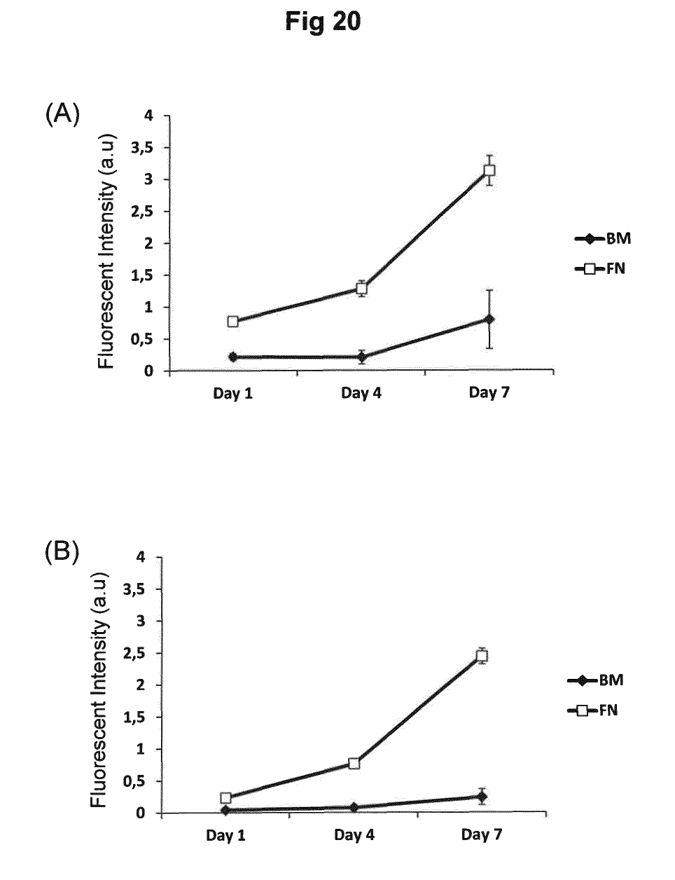

FIG. 20 shows growth curves (n=6, SEM; (A): 10 000 HUVEC/well; (B): 3 000 HUVEC/well) of proliferating cells (HUVEC) within a film of B. mori silk fibroin (“BM”, solid diamonds), compared to a corresponding film of FN_{cc}-RepCT (SEQ ID NO: 27; “FN”, open squares). Live staining further confirms the presence of viable cells, at day 8 within both film types (data not shown).

### Example 4

### Formulation of Silk Scaffolds with Integrated Human Pluripotent Stem Cells (hPSCs)

### Foam Formation

A 20 μl droplet of FN_{cc}-RepCT (SEQ ID NO: 27; 3 mg/ml) and laminin 521 (BioLamina, to a final concentration of 10 μg/ml) was placed at the center of a hydrophobic culture well. Air was pipetted into the droplet by quickly pipetting up and down 20 strokes with a pipette set at 40 μl, to create a dense wet foam. 50 000 hPSCs typically at 10 000 cell/μl concentration in Essential 8™ medium (Life Technologies) was immediately introduced into the foam by another 10 strokes to disperse the cells throughout the 3D structure. The cell-containing foam was then stabilized in a cell incubator at 37° C. for 20 min before the addition of 1 ml Essential 8™ containing ROCK inhibitor Y27632, 10 μM, suitable for a 24-well plate. The next day fresh culture medium was added without ROCK inhibitor, and medium was changed daily.

### Film Formation

Films were made by adding 10-20 μl of FN_{cc}-RepCT (SEQ ID NO: 27; 3 mg/ml) and laminin at the center of a hydrophobic well. The silk solution was formed into the desired shape and size using the pipette tip and typically 30 000 to 50 000 hPSCs (at least 10 000 cell/μl concentration) were added by gently dropping the solution into the center of the silk protein, letting the cells float out and immerse into the protein mix. The films were then stabilized in a cell incubator at 37° C. at 20-40 min depending on the size of the film before the addition of 0.5 ml (suitable for a 24-well plate) Essential 8™ medium containing ROCK inhibitor, 10 μm. The next day fresh culture medium was added without ROCK inhibitor and medium was changed daily. PSCs integrated in silk discs can easily be monitored by bright field microscopy and the time point for initiation of differentiation is decided when cells reach the confluence for the protocol of choice.

### Immunostaining for PSCs Included in Foam and Film

Immunocytochemistry was performed at selected time points after integration of cells in the silk. The silk scaffolds were washed once in PBS before the addition of 4% paraformaldehyde for 15 min. Permeabilization was carried out for 15 min in PBS with 0.1% Triton X-100 before blocking with 10% donkey serum (Jackson ImmunoResearch). Primary antibodies were incubated over night at 4° C. in PBS with 0.1% Tween-20 (PBS-T) and 5% serum. Secondary antibodies were incubated for 1 h at RT in PBS-T and 5% serum. Nuclei were counterstained using DAPI (Sigma) and incubated for 30 min. Samples were washed three times with PBS-T between each incubation.

Primary antibodies used: polyclonal goat anti-Nanog, 1:50 dilution (R&D), polyclonal rabbit anti-laminin, 1:200 dilution (Abcam).

Secondary antibodies used: donkey anti-rabbit 688 (Abcam) and donkey anti-goat 488 (Jackson ImmunoReasech) both at 1:1000 dilution.

Samples were imaged using a Leica DMI6000 B microscope and the image software ImageJ.

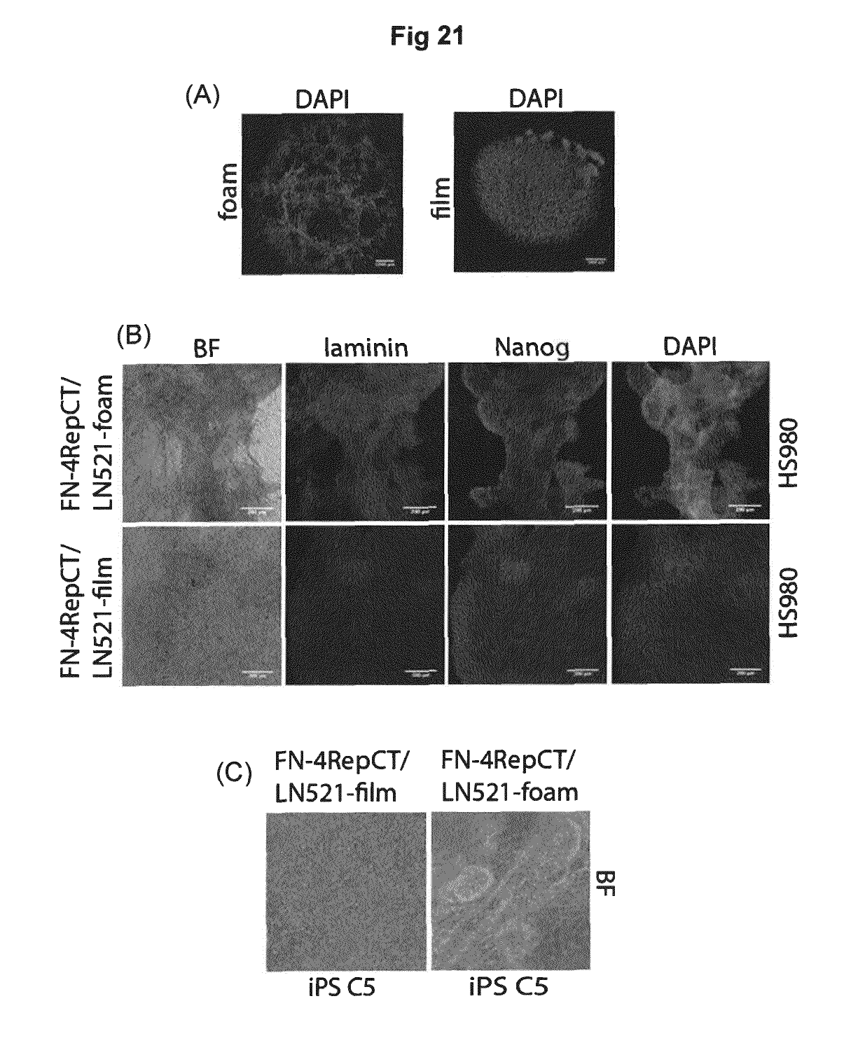

FIG. 21 shows cultivation of PSCs integrated into silk foam and film:
(A) Example micrographs of foam and film of FN_{cc}-RepCT (SEQ ID NO: 27) together with laminin 521 (LN521) at 24 h after inclusion of 50 000 human iPS C5. Cell distribution was visualized by nuclear DAPI stain (blue). Scale bars represent 1000 μm.
(B) Human embryonic cells, HS980 proliferated well and remained Nanog positive 72h after integration into foam (upper panel) and film (lower panel) of FN_{cc}-RepCT (SEQ ID NO: 27) silk as revealed by ICC. BF is brightfield. The laminin-coated silk was visualized by an anti-laminin antibody (Abcam) in green and pluripotency by anti-nanog (R&D) in red. Nuclei were counterstained with DAPI (blue). Scale bars represent 200 μm.
(C) Representative images of proliferating iPS C5 cells in FN_{cc}-RepCT (SEQ ID NO: 27) foam and film at 72 h after inclusion visualized with bright field microscopy.

Conclusions: Human pluripotent stem cells (hPSCs) such as embryonic stem cells (ESC) and induced pluripotent cells (iPS) survive and proliferate well after integration into foams and films of silk protein.

### Example 5

### Integration of Cells into Silk Films as Efficient Seeding Method

Two different cell types were tested: smooth muscle cells (human coronary artery, Gibco) and Human Umbilical Vein Endothelial Cells (Promocell). Cells suspended in respective culture media were mixed 1:1 with FN_{cc}-RepCT (SEQ ID NO: 27; 3 mg/ml) and then seeded as a drop in culture wells (either uncoated, or pre-coated with gelatin or silks made of RepCT (“WT”, SEQ ID NO: 2) or FN_{cc}-RepCT (“FN”, SEQ ID NO: 27).

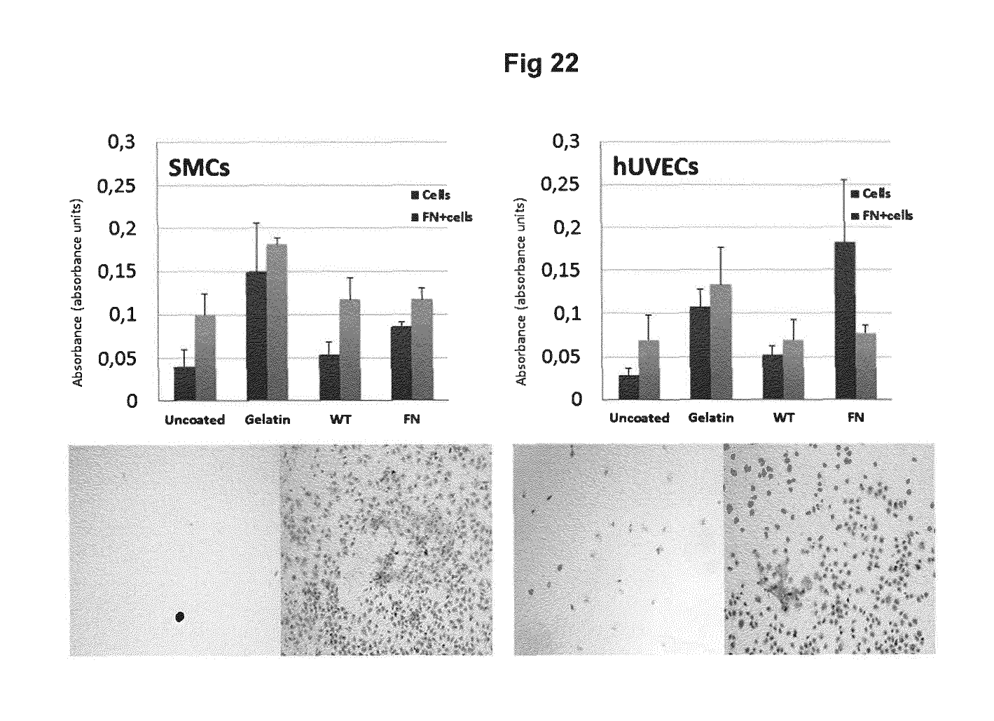

The amount of cells adhered within 30 min was analyzed after three subsequent washes before fixation and staining with Crystal violet. FIG. 22, upper row shows absorbance from the crystal violet stained cells after dissolved from being adhered to the culture well. Significantly more cells adhered to uncoated wells if seeded within a silk film. FIG. 22, lower row shows micrographs of the stained cells. The morphology of the adhered cells confirms proper attachment and spreading.

It is concluded that formulation of films with integrated cells provides a high seeding efficiency, yielding quickly and viably adhered cells.

### Example 6

### Differentiation of Stem Cells Integrated into Silk Scaffolds

Fibers and foam were prepared from FN_{cc}-RepCT (SEQ ID NO: 27) with integrated human mesenchymal stem cells (hMSC) as set out in Example 1.

### (A) Adipogenic or Osteogenic Differentiation

The macrostructures with integrated hSMC cells were subjected to either adipogenic or osteogenic differentiation medium (PromoCell) after 7 days of culture. Media was changed every third day until day 14. The samples were then subjected to fixation and staining with the lipid marker Red Oil O (Sigma Aldrich) for fat, and the osteogenic marker Alizarin Red S (Sigma Aldrich) for bone, all according to standard protocols.

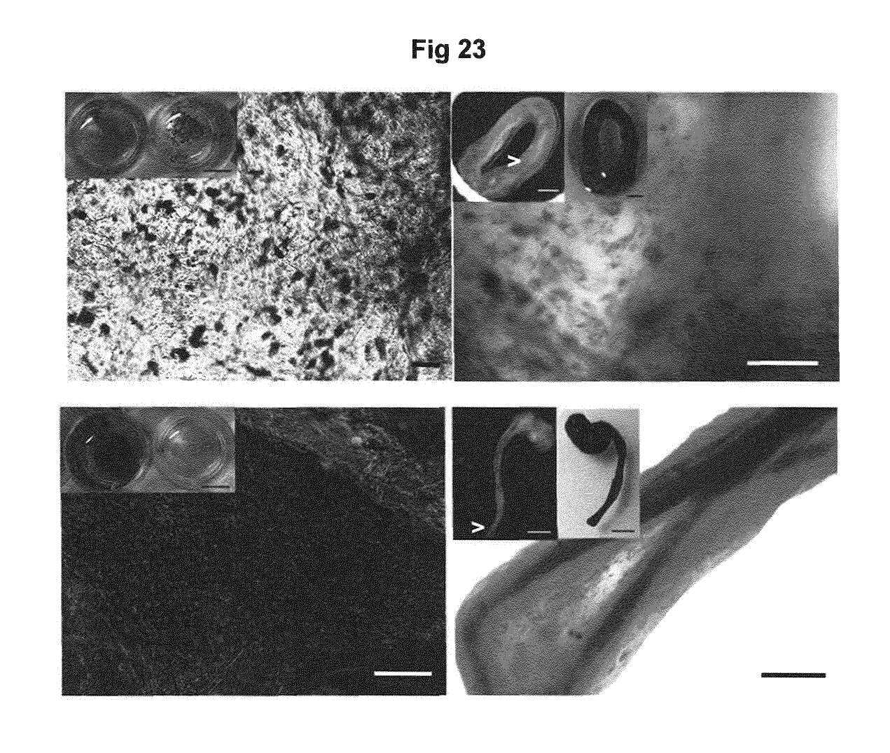

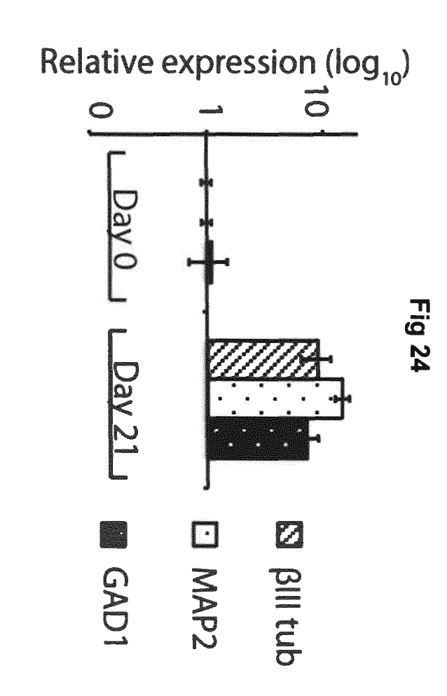

FIG. 23, upper row shows hMSCs differentiated into the adipogenic linage contains fat lipids, visualized by Red Oil staining of foams (left) and fibers (right). (N=2, n=4). Scale bars=100 μm. Insets show photos of foams (differentiated (left) and undifferentiated (right), scale bars=6.6 mm), and fibers (unstained (left), and Red oil stained (right), scale bars 1 mm). FIG. 23, lower row shows hMSCs differentiated into the osteogenic linage and probed with osteogenic marker for calcium content (Alizarin Red S (red)) in foam (upper left, scale bar=100 μm) and fiber (upper right, scale bar=200 μm). (N=2, n=4). Insets show photos of foams (differentiated (left) and undifferentiated (right), scale bars=6.6 mm), and fibers (unstained (left), and Alizarin Red S stained (right), scale bars=1 mm).

Lipid droplets were found throughout those silk foam and fibers with incorporated cells that had been treated with adipocyte induction media (FIG. 23, upper row). Calcium was found deposited throughout scaffolds treated with osteoblast induction media, with accumulation also in the innermost part of the fibers (FIG. 23, lower row).

### (B) Neuronal Differentiation

The macrostructures with integrated hSMC cells were cultured for 3 days and then subjected to dual-SMAD inhibition (Noggin and SB431542) for 7 days. This protocol yields neural progenitor cells. Thereafter, the medium was replaced to neuronal progenitor differentiation media, and the culture was continued for 14 days, followed by RT-qPCR analysis of the neuronal differentiation markers βIII tub, MAP2 and GAD1.

FIG. 24 shows relative gene expression analyzed by RT-qPCR of the neuronal progenitor markers βIII tub, MAP2 and GAD1 at day 0 and day 21. All data represent the mean ±SD for five independent cultivations (n=5).

It is concluded that human mesenchymal stem cells within the silk scaffolds are accessible for differentiation. Successful differentiation could be confirmed after fixation and staining with a lipid marker for fat, and an osteogenic marker for bone. Successful differentiation could also be confirmed after RT-qPCR analysis of neuronal differentiation markers.

### Example 7

### Cell Spreading Following Integration into Silk Scaffolds

In order to investigate the effect a fibrillar silk network have on cell spreading, macrostructures incorporating cells are prepared from FN_{cc}-RepCT (SEQ ID NO: 27) as set out in Example 1.

For comparison, the same cell type is seeded within a hydrogel of alginate with covalently coupled RGD motifs (NovaMatrix). The RGD alginate is prepared as 2% mixture in cell culture media together with cells, and submersion into CaCl₂ (100 mM) is used to trigger gelation.

Confocal reflection microscopy is used to collect high resolution 3D images of the native hydrated state of silk and hydrogel scaffolds with integrated cells.

The adhesion and spreading of cells integrated within the silk and hydrogel scaffolds is evaluated using laser scanning confocal microscopy. An inverted system equipped with fluorescence and phase contrast is used to allow visualization of both cells and material.

Immunohistochemistry is used to detect the important components (e.g. integrins, paxillin, vinculin, f-actin) of the various stages of adhesion (focal complexes, focal adhesions, fibrillar adhesion, 3D adhesions) at selected time points.

1. A method for the cultivation of eukaryotic cells, comprising the steps:
(a) providing an aqueous solution of a silk protein capable of assembling into a water-insoluble macrostructure, wherein the silk protein optionally contains a cell-binding motif;
(b) preparing an aqueous mixture of a sample of the eukaryotic cells with the silk protein, wherein the silk protein remains dissolved in the aqueous mixture;
(c) allowing the silk protein to assemble into a water-insoluble macrostructure in the presence of the eukaryotic cells, thereby forming a scaffold material for cultivating the eukaryotic cells; and
(d) maintaining the eukaryotic cells within the scaffold material under conditions suitable for cell culture.

2. The method according to claim 1, wherein the macrostructure is brought into a shape selected from fiber, foam, film, fiber mesh, capsules and nets.

3. The method according to claim 1, wherein the eukaryotic cells are selected from mammalian cells; and stem cells; or a combination of at least two different mammalian cell types.

4. The method according to claim 1, wherein the silk protein is a fibroin.

5. The method according to claim 1, wherein the silk protein is a spider silk protein.

6. The method according to claim 5, wherein the spider silk protein is comprising, or consisting of, the protein moieties REP and CT, wherein
REP is a repetitive fragment of from 70 to 300 amino acid residues, selected from the group consisting of L(AG)ₙL, L(AG)ₙAL, L(GA)ₙL, and L(GA)ₙGL, wherein
n is an integer from 2 to 10;
each individual A segment is an amino acid sequence of from 8 to 18 amino acid residues, wherein from 0 to 3 of the amino acid residues are not Ala, and the remaining amino acid residues are Ala;
each individual G segment is an amino acid sequence of from 12 to 30 amino acid residues, wherein at least 40% of the amino acid residues are Gly; and
each individual L segment is a linker amino acid sequence of from 0 to 30 amino acid residues; and
CT is a fragment of from 70 to 120 amino acid residues, having at least 70% identity to SEQ ID NO: 3 or SEQ ID NO: 68;
and wherein the optional cell-binding motif is arranged either terminally in the spider silk protein, or between the moieties, or within any of the moieties.

7. The method according to claim 1, wherein the silk protein contains a cell-binding motif selected from RGD, IKVAV (SEQ ID NO: 10), YIGSR (SEQ ID NO: 11), EPDIM (SEQ ID NO: 12), NKDIL (SEQ ID NO: 13), GRKRK (SEQ ID NO: 14), KYGAASIKVAVSADR (SEQ ID NO: 15), NGEPRGDTYRAY (SEQ ID NO: 16), PQVTRGDVFTM (SEQ ID NO: 17), AVTGRGDSPASS (SEQ ID NO: 18), TGRGDSPA (SEQ ID NO: 19), CTGRGDSPAC (SEQ ID NO: 20) and FN_{cc} (SEQ ID NO: 9);
wherein FN_{cc} is C¹X¹X²RGDX³X⁴X⁵C²;
wherein each of X¹, X², X³, X⁴ and X⁵ are independently selected from natural amino acid residues other than cysteine; and C¹ and C² are connected via a disulphide bond.

8. A process for manufacturing a cell culture product comprising (i) a scaffold material for cultivating eukaryotic cells; and (ii) eukaryotic cells, which are growing integrated with the scaffold material, comprising the steps:
(a) providing an aqueous solution of a silk protein capable of assembling into a water-insoluble macrostructure, wherein the silk protein optionally contains a cell-binding motif;
(b) preparing an aqueous mixture of a sample of the eukaryotic cells with the silk protein, wherein the silk protein remains dissolved in the aqueous mixture; and
(c) allowing the silk protein to assemble into a water-insoluble macrostructure in the presence of the eukaryotic cells, thereby forming the scaffold material for cultivating the eukaryotic cells.

9. The process for manufacturing a cell culture product according to claim 8,
wherein the macrostructure is brought into a shape selected from fiber, foam, film, fiber mesh, capsules and nets; and/or
wherein the eukaryotic cells are selected from mammalian cells; and stem cells; or a combination of at least two different mammalian cell types; and/or
wherein the silk protein is a fibroin or a spider silk protein.

10. A cell culture product comprising (i) a scaffold material for cultivating eukaryotic cells, which is a water-insoluble macrostructure of a silk protein capable of assembling into a water-insoluble macrostructure, wherein the silk protein optionally contains a cell-binding motif; and (ii) eukaryotic cells, which are growing integrated with the scaffold material.

11. A cell culture product comprising (i) a scaffold material for cultivating eukaryotic cells, which is a water-insoluble macrostructure of a silk protein capable of assembling into a water-insoluble macrostructure, wherein the silk protein optionally contains a cell-binding motif; and (ii) eukaryotic cells, which are growing integrated with the scaffold material, wherein said cell culture product is obtainable or obtained by the process according to claim 8.

12-13. (canceled)

14. The method according to claim 1, wherein the aqueous mixture of step (b) further contains cell-binding proteins or polypeptides.

15. The cell culture product according to claim 10,
wherein the macrostructure is brought into a shape selected from fiber, foam, film, fiber mesh, capsules and nets; and/or
wherein the eukaryotic cells are selected from mammalian cells; and stem cells; or a combination of at least two different mammalian cell types; and/or
wherein the silk protein is a fibroin or a spider silk protein.

16. The method according to claim 2, wherein the macrostructure is brought into a shape selected fiber or foam.

17. The method according to claim 3, wherein the mammalian cells are selected from primary cells and cell lines, and the stem cells are mesenchymal stem cells.

18. The method according to claim 17, wherein the mammalian cells are endothelical cells, fibroblasts, keratinocytes, skeletal muscle satellite cells, skeletal muscle myoblasts, Schwann cells, pancreatic β-cells, pancreatic islet cells, hepatocytes and glioma-forming cells.

19. The method according to claim 4, wherein the fibroin is a silkworm fibroin.

20. The method according to claim 6, wherein the optional cell-binding motif is arranged terminally in the spider silk protein.

21. The method according to claim 7, wherein the cell-binding motif is selected from FN_{cc}, GRKRK, IKVAV, RGD and CTGRGDSPAC.

22. The method according to claim 7, wherein the cell-binding motif is selected from FN_{cc} and CTGRGDSPAC.
